(19) United States
(12) Patent Application Publication
(10) Pub. No.: US 2016/0355449 A1
(43) Pub. Date: Dec. 8, 2016

(54) Hydrogenation Using Highly Selective Catalyst

(71) Applicant: Chevron Phillips Chemical Company LP, The Woodlands, TX (US)

(72) Inventors: Timothy O. Odi, Kingwood, TX (US); Zongxuan Hong, Houston, TX (US); Joseph Bergmeister, III, Kingwood, TX (US); Tin-Tack Peter Cheung, Kingwood, TX (US); Charles D. Nolidin, Singapore (SG); Thomas J. Gonzales, Houston, TX (US); Jennifer L. Nill, Dickinson, TX (US); David W. Dockter, Kingwood, TX (US)

(21) Appl. No.: 14/733,595

(22) Filed: Jun. 8, 2015

(51) Int. Cl. C07C 5/05; C07C 4/02

(52) U.S. Cl. CPC C07C 5/05; C07C 4/02

(57) ABSTRACT

A process comprising hydrogenating a highly unsaturated hydrocarbon in the presence of a first hydrogenation catalyst and a second hydrogenation catalyst to one or more compounds including an unsaturated hydrocarbon such that a total conversion of the highly unsaturated hydrocarbon is about 99 mol % or greater. In the process, the first hydrogenation catalyst, the second hydrogenation catalyst, or both, have a hydrogenation selectivity to the unsaturated hydrocarbon of about 90% or greater.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### FIELD

The present disclosure relates to the production of an unsaturated hydrocarbon, and more particularly to a hydrogenation of compounds using highly selective catalyst.

### BACKGROUND

Unsaturated hydrocarbons such as ethylene and propylene are often employed as feedstocks in preparing value added chemicals and polymers. Unsaturated hydrocarbons can be produced by pyrolysis or cracking of hydrocarbons including hydrocarbons derived from coal, oil, gas, synthetic crude, naphthas, refinery gases, ethane, propane, butane, and the like. Unsaturated hydrocarbon products produced in these manners usually contain highly unsaturated hydrocarbons such as acetylenes and diolefins that can adversely affect the production of subsequent chemicals and polymers. Thus, to form an unsaturated hydrocarbon product such as a polymer grade monoolefin, the amount of acetylenes and diolefins in the monoolefin stream can be typically reduced.

One technique commonly used to reduce the amount of acetylenes and diolefins in an unsaturated hydrocarbon stream primarily comprising monoolefins involves selectively hydrogenating the acetylenes and diolefins to monoolefins. This process is selective in that hydrogenation of the monoolefin and the highly unsaturated hydrocarbon to the saturated hydrocarbons is minimized. For example, the hydrogenation of ethylene or acetylene to ethane is minimized.

One challenge to the selective hydrogenation process is the potential for a runaway reaction leading to the uncontrolled hydrogenation of ethylene to ethane. One methodology to minimize runaway reactions is to use a highly selective hydrogenation catalyst. The availability of highly selective hydrogenation catalysts, however, has brought about other challenges for converting highly unsaturated hydrocarbons to unsaturated hydrocarbons.

### SUMMARY

Disclosed herein is a process comprising hydrogenating a highly unsaturated hydrocarbon in the presence of a first hydrogenation catalyst to yield an unsaturated hydrocarbon, a saturated hydrocarbon, and an unconverted highly unsaturated hydrocarbon, wherein a conversion of the highly unsaturated hydrocarbon to the unsaturated hydrocarbon and the saturated hydrocarbon in the presence of the first hydrogenation catalyst is about 90 mol % or greater, and hydrogenating the unconverted highly unsaturated hydrocarbon in the presence of a second hydrogenation catalyst to yield the unsaturated hydrocarbon and the saturated hydrocarbon, and the unconverted highly unsaturated hydrocarbon, wherein a total conversion of the highly unsaturated hydrocarbon to the unsaturated hydrocarbon and the saturated hydrocarbon after hydrogenation in the presence of the first hydrogenation catalyst and after hydrogenation in the presence of the second hydrogenation catalyst is about 99 mol % or greater, wherein the first hydrogenation catalyst, the second hydrogenation catalyst, or both, have a hydrogenation selectivity to the unsaturated hydrocarbon of about 90% or greater based on the moles of the highly unsaturated hydrocarbon which are converted.

Also disclosed herein is a system comprising a hydrocarbon stream comprising a highly unsaturated hydrocarbon, an unsaturated hydrocarbon, and optionally, a saturated hydrocarbon; a first reaction zone comprising a first hydrogenation catalyst, wherein the hydrocarbon stream contacts the first hydrogenation catalyst in the first reaction zone, and wherein at least a portion of the highly unsaturated hydrocarbon from the hydrocarbon stream is hydrogenated in the first reaction zone; and a second reaction zone comprising a second hydrogenation catalyst, wherein the second reaction zone receives a first effluent stream comprising the unsaturated hydrocarbon, an unconverted highly unsaturated hydrocarbon, and optionally, the saturated hydrocarbon from the first reaction zone, wherein at least a portion of the unconverted highly unsaturated hydrocarbon is hydrogenated in the second reaction zone, wherein conversion of the highly unsaturated hydrocarbon to the unsaturated hydrocarbon and the saturated hydrocarbon after hydrogenation in the first reaction zone is about 90 mol % or greater based on moles of the highly unsaturated hydrocarbon in the hydrocarbon stream, wherein a total conversion of the highly unsaturated hydrocarbon to the unsaturated hydrocarbon and the saturated hydrocarbon after hydrogenation in the first and second reaction zones is about 99 mol % or greater based on moles of the highly unsaturated hydrocarbon in the hydrocarbon stream, and wherein the first hydrogenation catalyst, the second hydrogenation catalyst, or both have a hydrogenation selectivity to the unsaturated hydrocarbon of about 90 mol % or greater based on the moles of highly unsaturated hydrocarbon which are converted.

Further disclosed herein is a process comprising cracking a feed stream to produce a cracked gas stream comprising acetylene, ethylene, ethane, methane, and C₃⁺ components, hydrogenating acetylene in the presence of a first hydrogenation catalyst in a first reaction zone, wherein conversion of acetylene to ethylene and ethane in the first reaction zone is about 90 mol % or greater of the total acetylene fed to the first reaction zone, receiving a first effluent stream from the first reaction zone into a second reaction zone, wherein the first effluent stream can comprise unconverted acetylene, hydrogenating the unconverted acetylene of the first effluent stream in the presence of a second hydrogenation catalyst in the second reaction zone, wherein a total conversion of acetylene to ethylene and ethane after hydrogenation in the first reaction zone and the second reaction zone is about 99 mol % or greater of the total acetylene fed to the first reaction zone, recovering a second effluent stream from the second reaction zone, removing ethylene from the second effluent stream to yield an ethylene stream, and polymerizing ethylene from the ethylene stream into one or more polymer products, wherein the first hydrogenation catalyst, the second hydrogenation catalyst, or both, have a hydrogenation selectivity to ethylene of about 90 mol % or greater based on moles of acetylene which are converted.

Further disclosed herein is a process comprising providing a first reaction zone comprising a first hydrogenation catalyst and a second reaction zone comprising a second hydrogenation catalyst, wherein the second reaction zone is fluidly connected to and downstream of the first reaction zone, wherein at least one of the first hydrogenation catalyst and the second hydrogenation catalyst can comprise a hydrogenation catalyst, and optionally, an organophosphorus compound, providing a highly unsaturated hydrocarbon to the first reaction zone, hydrogenating, in the first reaction zone, the highly unsaturated hydrocarbon to yield an unsaturated hydrocarbon, a saturated hydrocarbon, and an unconverted highly unsaturated hydrocarbon, wherein conversion of the highly unsaturated hydrocarbon to the unsaturated hydrocarbon and the saturated hydrocarbon after hydrogenation in the first reaction zone is about 90 mol % or greater based on moles of the highly unsaturated hydrocarbon provided to the first reaction zone, and hydrogenating, in the second reaction zone, the unconverted highly unsaturated hydrocarbon to yield the unsaturated hydrocarbon and the saturated hydrocarbon, wherein a total conversion of the highly unsaturated hydrocarbon to the unsaturated hydrocarbon and the saturated hydrocarbon after hydrogenation in the first reaction zone and the second reaction zone is about 99 mol % or greater based on moles of the highly unsaturated hydrocarbon provided to the first reaction zone, wherein at least one of the first hydrogenation catalyst and the second hydrogenation catalyst can comprise a hydrogenation selectivity to the unsaturated hydrocarbon of about 90 mol % or greater based on moles of the highly unsaturated hydrocarbon which are converted.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 illustrates embodiments of the disclosed system and process.

FIG. 2 illustrates other embodiments of the disclosed system and process.

FIG. 3 illustrates further embodiments of the disclosed system and process.

FIG. 4 shows the inlet acetylene to ethylene mole ratio in a first reactor of a kinetic model and its effect on hydrogenation conversion of acetylene.

### DETAILED DESCRIPTION

Embodiments of systems and processes for hydrogenation using highly selective catalysts are disclosed herein. It should be understood at the outset that although an illustrative implementation of one or more embodiments are provided below, the disclosed systems and/or processes can be implemented using any number of techniques, whether currently known or in existence. The disclosure should in no way be limited to the illustrative implementations, drawings, and techniques illustrated below, including the exemplary designs and implementations illustrated and described herein, but can be modified within the scope of the appended claims along with their full scope of equivalents.

As used herein, a “highly unsaturated hydrocarbon” is defined as a hydrocarbon containing a triple bond, two conjugated carbon-carbon double bonds, or two cumulative carbon-carbon double bonds. Examples of a highly unsaturated hydrocarbon include, but are not limited to, alkynes such as acetylene, methylacetylene (also referred to as propyne), and butynes; diolefins such as propadiene, butadienes, pentadienes (including isoprene); and the like and combinations thereof.

As used herein, an “unsaturated hydrocarbon” is defined as a hydrocarbon containing an isolated carbon-carbon double bond. Examples of an unsaturated hydrocarbon include, but are not limited to, monoolefins such as ethylene, propylene, butenes, pentenes, and the like and combinations thereof.

As used herein, a “saturated hydrocarbon” is defined as a hydrocarbon containing no carbon-carbon double bonds, conjugated carbon-carbon double bonds, cumulative carbon-carbon double bonds, or carbon-carbon triple bonds. Examples of a saturated hydrocarbon include, but are not limited to, methane, ethane, propane, butanes, pentanes, and the like and combinations thereof.

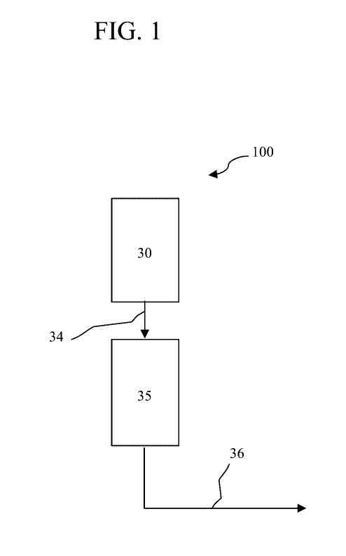

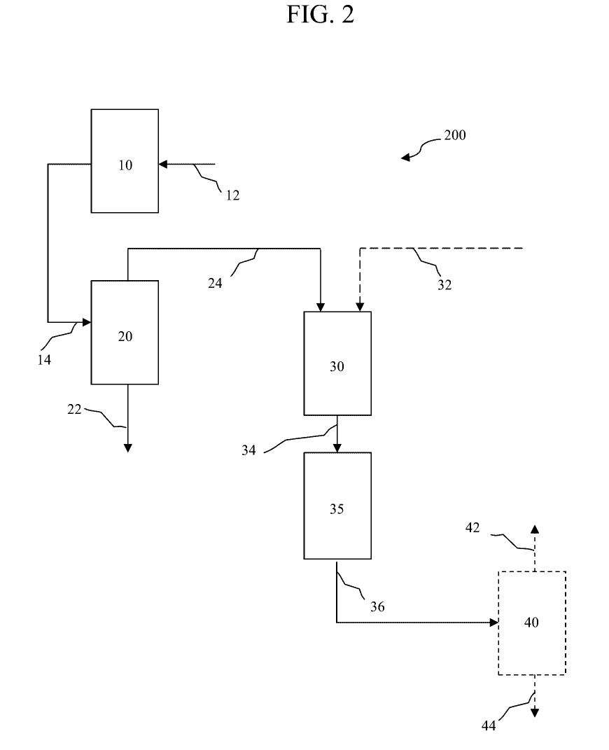

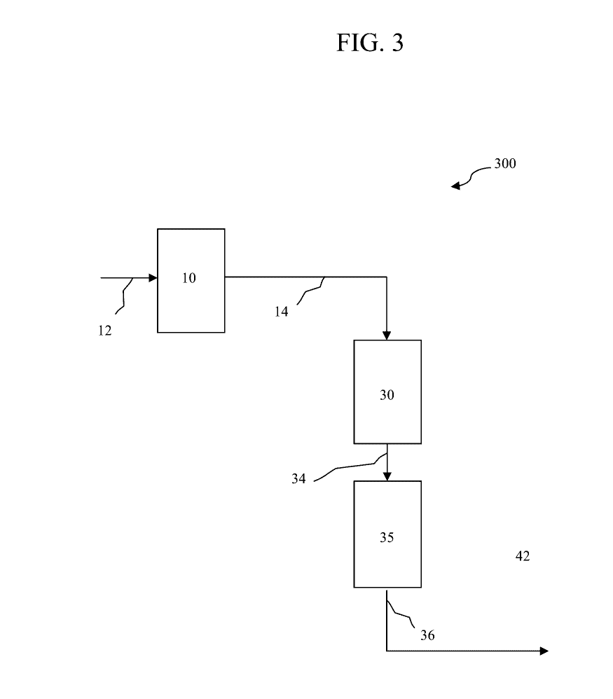

FIG. 1 shows embodiments of the disclosed system 100 which can comprise a first reaction zone 30, a second reaction zone 35, a first effluent stream 34 (or flow path 34) via which a first effluent flows from the first reaction zone 30 to a second reaction zone 35, and a second effluent stream 36 via which a second effluent flows from the second reaction zone 35. In the first reaction zone 30 and the second reaction zone 35, at least one highly unsaturated hydrocarbon received from a hydrocarbon stream or a cracked gas stream (e.g., stream 24 of FIG. 2 or stream 14 of FIG. 3, described in detail below) is hydrogenated in the presence of a first hydrogenation catalyst and a second hydrogenation catalyst, respectively, (such as those embodiments which are described herein) to yield one or more compounds comprising an unsaturated hydrocarbon, a saturated hydrocarbon, an unconverted highly unsaturated hydrocarbon, or combinations thereof. Generally, a highly unsaturated hydrocarbon is hydrogenated in the first reaction zone 30, and unconverted or unreacted highly unsaturated hydrocarbon flows in the first effluent stream (e.g., via first effluent stream 34 or flow path 34) to the second reaction zone 35, wherein the unconverted highly unsaturated hydrocarbon is hydrogenated in the second reaction zone 35 to yield one or more compounds comprising the unsaturated hydrocarbon, the saturated hydrocarbon, the unconverted highly unsaturated hydrocarbon, or combinations thereof. Hydrogenation generally occurs in the first reaction zone 30 and second reaction zone 35 in the presence of a first hydrogenation catalyst and a second hydrogenation catalyst, respectively, (embodiments which are described herein) and in the presence of hydrogen.

In embodiments, the first reaction zone 30 can comprise a first reactor, and the second reaction zone 35 can comprise a second reactor. In such embodiments, the first reactor can be separate from and in series with the second reactor (e.g., the first reactor and second reactor are connected in series). In such embodiments, the first effluent can flow in a first effluent stream 34 which fluidly connects the first reaction zone 30 comprising the first reactor and the second reaction zone 35 comprising the second reactor such that the effluent of the first reaction zone 30 flows to the second reaction zone 35 (e.g., second reaction zone 35 is downstream from first reaction zone 30). In embodiments, first effluent stream 34 can comprise equipment (e.g., pipe, valves, pumps, heat exchangers, instrumentation, other equipment known in the art with the aid of this disclosure, or combinations thereof). In embodiments, a heat exchanger can be placed between the first reaction zone 30 and the second reaction zone 35 to add or remove heat to achieve the conversions disclosed herein in the second reaction zone 35, for example, due to the high selectivity of the first hydrogenation catalyst in the first reaction zone 30. In embodiments, no heat is added to the first effluent stream 34 between the first reaction zone 30 and the second reaction zone 35. In embodiments, a first temperature of the first effluent stream 34 as the first effluent stream 34 flows into the second reaction zone 35 is the same as or lower than a second temperature of the first effluent stream 34 as the first effluent stream 34 flows from the first reaction zone 30.

In embodiments, the first reaction zone 30 can be contained within the same vessel as the second reaction zone 35 (e.g., first reaction zone 30 and second reaction zone 35 can comprise catalyst beds of the same reactor). In such embodiments, the first flow path 34 can comprise equipment (e.g., pipe, values, baffles, screens, packing, other internal equipment known in the art with the aid of this disclosure, or combinations thereof), which fluidly connects the first reaction zone 30 and the second reaction zone 35 such that the effluent can flow from the first reaction zone 30 to the second reaction zone 35 (e.g., the first reaction zone 30 and the second reaction zone 35 are fluidly connected in series).

In additional or alternative embodiments, the first reaction zone 30, the second reaction zone 35, or both can represent a plurality of reactors. The plurality of reactors of the first reaction zone 30, the second reaction zone 35, or both can optionally be separated by a means to remove heat produced by the reaction. The plurality of reactors of the first reaction zone 30 can be configured in parallel, in series, or both. Likewise, the plurality of reactors of the second reaction zone 30 can be configured in parallel, in series, or both. The plurality of reactors the first reaction zone 30, the second reaction zone 35, or both can optionally be separated by a means to control inlet and effluent flows from reactors or heat removal means allowing for individual or alternatively groups of reactors within the plurality of reactors to be regenerated.

The first and second hydrogenation catalysts can be arranged in any suitable configuration within the first reaction zone 30 and the second reaction zone 35, such as a fixed catalyst bed, a fluidized bed, or both.

In embodiments, the temperature within the first reaction zone 30, the second reaction zone 35, or both, can be in the range of from about 5° C. to about 300° C.; alternatively, from about 10° C. to about 250° C.; alternatively, from about 15° C. to about 200° C. In some embodiments, the pressure within the first reaction zone 30, the second reaction zone 35, or both, can be in the range of from about 15 (204 kPa) to about 2,000 (13,890 kPa) pounds per square inch gauge (psig); alternatively, from about 50 psig (446 kPa) to about 1,500 psig (10,443 kPa); alternatively, from about 100 psig (790 kPa) to about 1,000 psig (6,996 kPa).

FIGS. 2 and 3 show additional embodiments of the disclosed system.

In each of FIGS. 2 and 3, embodiments of the systems 200 and 300 can include a furnace 10 comprising one or more metal tubes into which the components of a feed stream 12 (e.g., hydrocarbon feedstock) flow. The tubes of the furnace 10 are configured to thermally crack at least one of the hydrocarbon components of the feed stream 12 (e.g., comprising a raw gas, oil and gas, oil, petroleum naphtha, a refinery stream comprising a crackable hydrocarbon, and other feed sources known in the art with the aid of this disclosure). In embodiments, the furnace 10 can comprise an insulated box and one or more burners. The components of the feed stream 12 can be heated (e.g., by burning of a fuel in the burner) as the components flow through the one or more tubes in the furnace 10 such that at least one component of the feed stream 12 is thermally cracked, for example, to produce an unsaturated hydrocarbon (e.g., ethylene, propylene, or both). The cracked gas stream 14 emits from the furnace 10.

In embodiments of the system 200 in FIG. 2, the first reaction zone 30 and the second reaction zone 35 can comprise one or more hydrogenation reactors that belong to an acetylene removal unit (ARU) of an unsaturated hydrocarbon production plant in a frontend deethanizer configuration, a frontend depropanizer configuration, or a backend configuration (described in detail below).

Embodiments of the system 200 in FIG. 2 can include a fractionation zone 20 which is upstream of the first reaction zone 30 and the second reaction zone 35. The fractionation zone 20 can comprise a vessel having internal components such as distillation trays (e.g., sieve-type, dual-flow, bubble cap, donut), packing materials, or both. The fractionation zone 20 can operate at conditions that provide for the fractionation of a cracked gas stream according to the embodiments disclosed herein.

In embodiments, the fractionation zone 20 can comprise a deethanizer (the system 200 is in a frontend deethanizer configuration), a depropanizer (the system 200 is in a frontend depropanizer configuration), or both a demethanizer and a deethanizer (the system 200 is in a backend configuration).

The fractionation zone 20 comprising a deethanizer can receive a cracked gas stream 14 from an unsaturated hydrocarbon production process (e.g., from the furnace 10) and fractionate the cracked gas stream 14 into an overhead product (e.g., a C₂⁻ stream) and a bottoms product (e.g., a C₃⁺ stream). In such embodiments, the cracked gas stream 14 can comprise hydrogen, carbon monoxide, propane, ethane, methane, methylacetylene, propadiene, acetylene, ethylene, propylene, C₄⁺ components (e.g., C₄ hydrocarbons and heavier), or combinations thereof. The overhead product can be an ethane-rich stream; the overhead product can comprise about 90 mol % or greater of the ethane contained in the cracked gas stream; the overhead product can comprise C₂⁻ components such as acetylene, ethylene, ethane, methane, hydrogen, carbon monoxide, or combinations thereof; or combinations thereof. In embodiments, the overhead product can be fed to the first reaction zone 30, the second reaction zone 35, or both, via one or more streams such as hydrocarbon stream 24. The bottoms product (e.g., comprising C₃⁺ components such as propane, methylacetylene, propadiene, propylene, or combinations thereof) can flow from the fractionation zone 20 via stream 22.

The fractionation zone 20 comprising a depropanizer can receive a cracked gas stream 14 from an unsaturated hydrocarbon production process (e.g., from the furnace 10) and fractionate the cracked gas stream 14 into an overhead product (e.g., a C₃⁻ stream) and a bottoms product (e.g., a C₄⁺ stream). In such embodiments, the cracked gas stream 14 can comprise hydrogen, carbon monoxide, propane, ethane, methane, methylacetylene, propadiene, acetylene, ethylene, propylene, C₄⁺ components (e.g., C₄ hydrocarbons and heavier), or combinations thereof. The overhead product can comprise about 90 mol % or greater of the ethane and/or propane contained in the cracked gas stream 14. The overhead product (e.g., comprising C₃⁻ components such as hydrogen, carbon monoxide, propane, ethane, methane, methylacetylene, propadiene, acetylene, ethylene, propylene, or combinations thereof) can be fed to the first stage 30, the second stage 35, or both, via one or more streams such as hydrocarbon stream 24. The bottoms product (e.g., comprising C₄⁺ components such as C₄ hydrocarbons and heavier) can flow from the fractionation zone 20 via stream 22.

In embodiments, the fractionation zone 20 can comprise a demethanizer and a deethanizer. In such an embodiment, the demethanizer can receive a cracked gas stream 14 from an unsaturated hydrocarbon production process (e.g., from the furnace 10) and fractionate the cracked gas stream 14 into an overhead product (e.g., a methane-rich stream) and a bottoms product (e.g., a C₂⁺ stream). In such embodiments, the cracked gas stream 14 fed to the demethanizer can comprise hydrogen, carbon monoxide, propane, ethane, methane, methylacetylene, propadiene, acetylene, ethylene, propylene, C₄⁺ components (e.g., C₄ hydrocarbons and heavier), or combinations thereof. The overhead product of the demethanizer can comprise methane, hydrogen, and carbon monoxide; can comprise about 90 mol % or greater of the methane contained in the cracked gas stream 14; or both. The bottoms product of the demethanizer can comprise about 90 mol % or greater of the ethane contained in the cracked gas stream 14; the bottoms product of the demethanizer can comprise C₂⁺ components (e.g., ethane, acetylene, ethylene, methylacetylene, propadiene, propylene, propane, or combinations thereof). The bottoms product of the demethanizer then can flow to the deethanizer where the deethanizer fractionates the demethanizer bottoms products into an overhead product (e.g., a C₂⁻ stream) and a bottoms product (e.g., a C₃⁺ stream). The overhead product of the deethanizer can be an ethane-rich stream; the overhead product of the deethanizer can comprise about 90 mol % or greater of the ethane contained in the demethanizer bottoms product; the overhead product can comprise C₂⁻ components such as acetylene, ethylene, ethane, or combinations thereof. In embodiments, the overhead product of the deethanizer can be fed to the first reaction zone 30, the second reaction zone 35, or both, via one or more streams such as hydrocarbon stream 24. The bottoms product of the deethanizer (e.g., comprising C₃⁺ components such as propane, methylacetylene, propadiene, propylene, or combinations thereof) can flow from the fractionation zone 20 via stream 22.

Designated with dashed lines in FIG. 2, embodiments of system 200 in a frontend deethanizer configuration can include a fractionation zone 40 which is downstream of the second reaction zone 35 and which can receive the second effluent stream 36 from the second reaction zone 35. A saturated hydrocarbon stream 42 (designated with a dashed line) can emit from the fractionation zone 40, and an unsaturated hydrocarbon stream 44 (also designated with a dashed line) can emit from the fractionation zone 40. In such embodiments, the fractionation zone 40 can split (e.g., separate) an unsaturated hydrocarbon (e.g., ethylene, propylene) from a saturated hydrocarbon (e.g., ethane, propane) which are received from the second reaction zone 35 via second effluent stream 36. Unsaturated hydrocarbon (e.g., ethylene, propylene) in stream 44 (e.g., an ethylene stream or propylene stream) can be used in a polymerization process for the production of one or more polymer products.

In embodiments of system 200 in a frontend deethanizer configuration, the fractionation zone 40 can operate at conditions (e.g., temperatures and pressures) which separate components of the second effluent stream 36 such that unsaturated hydrocarbon flows through unsaturated hydrocarbon stream 44 and saturated hydrocarbon flows through saturated hydrocarbon stream 42. The fractionation zone 40 can comprise a vessel in which a suitable technique can be used to separate the unsaturated hydrocarbon and saturated hydrocarbon.

In embodiments of the system 300 in FIG. 3, the first reaction zone 30 and the second reaction zone 35 can comprise one or more hydrogenation reactors that belong to an acetylene removal unit (ARU) of an unsaturated hydrocarbon production plant in a raw gas configuration. In embodiments of the system 300 in a raw gas configuration, the cracked gas stream 14 can feed to the first reaction zone 30, the second reaction zone 35, or both, without first passing through a fractionation zone. In such raw gas configurations, the cracked gas stream 14 comprising hydrogen, carbon monoxide, propane, ethane, methane, methylacetylene, propadiene, acetylene, ethylene, propylene, C₄⁺ components (e.g., C₄ hydrocarbons and heavier), or combinations thereof, can feed directly to the first reaction zone 30, the second reaction zone 35, or both.

It is understood that first reaction zone 30 and second reaction zone 35, and likewise the first and second hydrogenation catalysts disclosed herein, are not limited to use in raw gas feed, frontend deethanizer, frontend depropanizer, or backend ARU feed configurations, and can be used in any process wherein a highly unsaturated hydrocarbon contained within a hydrocarbon stream is selectively hydrogenated to an unsaturated hydrocarbon.

Designated with a dashed line in FIG. 2, hydrogen, carbon monoxide, or both (e.g. via a makeup stream 32) can be fed to the first reaction zone 30, for example, in the system 200 having a back end configuration. In additional or alternative embodiments, hydrogen, carbon monoxide, or both can be fed to the second reaction zone 35. In embodiments having a makeup stream 32, the hydrocarbon stream 24 and makeup stream 32 can be combined in a single stream that is fed to first reaction zone 30. In additional or alternative embodiments, hydrogen and carbon monoxide can be fed to first reaction zone 30 or can be combined with hydrocarbon stream 24 via separate streams for each of hydrogen and carbon monoxide (e.g., a hydrogen stream and a carbon monoxide stream). In embodiments, hydrogen can be present in the system 200 in excess amounts. In an embodiment, the amount of carbon monoxide fed to first reaction zone 30 can comprise less than about 0.15 mol % based on the total moles of fluid being fed to the first reaction zone 30.

In embodiments, the systems 100, 200, and 300 can additionally comprise any equipment associated with hydrogenation processes, such as but not limited to, one or more pumps, one or more control devices, one or more measurement instruments (e.g., thermocouples, transducers, and flow meters), alternative inlet and/or outlet lines, one or more valves, one or more reboilers, one or more condensers, one or more accumulators, one or more tanks, one or more filters, one or more compressors, one or more dryers, or combinations thereof.

The feed stream 12 shown in the systems 100, 200, and 300 of FIGS. 1-3 can comprise any hydrocarbon feed source known in the art with the aid of this disclosure (e.g., natural gas liquid, ethane, propane, oil, petroleum naphtha, a refinery stream comprising a crackable hydrocarbon, or combinations thereof).

The cracked gas stream 14 shown in the systems 100, 200, and 300 of FIGS. 1-3 can comprise a highly unsaturated hydrocarbon, an unsaturated hydrocarbon, a saturated hydrocarbon, carbon monoxide, carbon dioxide, hydrogen, or combinations thereof. In embodiments, the cracked gas stream 14 can comprise from about 10 ppm to about 20,000 ppm of the highly unsaturated hydrocarbon based on the total weight of all hydrocarbons in the cracked gas stream 14.

The hydrocarbon stream 24 shown in FIG. 2 can comprise a highly unsaturated hydrocarbon, an unsaturated hydrocarbon, a saturated hydrocarbon, hydrogen, carbon monoxide, or combinations thereof.

The reactive and inert components within the first reaction zone 30 and/or second reaction zone 35 can collectively be referred to as a reaction medium. The amount (e.g., moles, weight, mass, flow, concentration (for example, in mol %, wt. %, mole ratio, or other means for determining concentration), other indicator of amount, or combinations thereof) of the components of the reaction medium within the first reaction zone 30 and second reaction zone 35 can change over time and/or can depend on the location of the reaction medium within the first reaction zone 30 and/or the second reaction zone 35. Generally, as hydrogenation occurs in the first reaction zone 30, the amount of the highly unsaturated hydrocarbon in the reaction medium decreases in the first reaction zone 30. After the reaction medium flows from the first reaction zone 30 to the second reaction zone 35 via flow path 34, the amount of the highly unsaturated hydrocarbon in the reaction medium further decreases as hydrogenation occurs in the second reaction zone 35. Conversely, as hydrogenation occurs in the first reaction zone 30, the amount of the unsaturated hydrocarbon (and optionally a saturated hydrocarbon) in the reaction medium increases in the first reaction zone 30. After the reaction medium flows from the first reaction zone 30 to the second reaction zone 35 via flow path 34, the amount of the unsaturated hydrocarbon (and optionally a saturated hydrocarbon) in the reaction medium further increases as hydrogenation occurs in the second reaction zone 35.

In embodiments, the reaction medium, depending on its location within the system 100 can comprise an unsaturated hydrocarbon, a unsaturated hydrocarbon, a saturated hydrocarbon, hydrogen, or combinations thereof.

For example, within the first reaction zone 30, the reaction medium can comprise an unsaturated hydrocarbon which is the product of the hydrogenation of a highly unsaturated hydrocarbon in the first reaction zone 30; an unsaturated hydrocarbon which was originally contained in the hydrocarbon stream 24 and is not the product of hydrogenation in the first reaction zone 30; and a highly unsaturated hydrocarbon which was originally contained in the hydrocarbon stream 24 and is unreacted or unconverted in the first reaction zone 30. Additionally, the reaction medium within the first reaction zone 30 can comprise a saturated hydrocarbon which is a side product of the hydrogenation reaction in the first reaction zone 30; the saturated hydrocarbon which was originally contained in the hydrocarbon stream 24; hydrogen fed to the first reaction zone 30 via stream 32; hydrogen which was originally contained in the hydrocarbon stream 24; or combinations thereof.

The first effluent stream 34 can comprise an unsaturated hydrocarbon which is the product of the hydrogenation of the highly unsaturated hydrocarbons in the first reaction zone 30, an unsaturated hydrocarbon which was originally contained in the hydrocarbon stream 24 and is not the product of hydrogenation in the first reaction zone 30. Additionally, the first effluent stream 34 can comprise a highly unsaturated hydrocarbon which was originally contained in the hydrocarbon stream 24 and is unreacted or unconverted in the first reaction zone 30; a saturated hydrocarbon which can be a side product of the hydrogenation reaction in the first reaction zone 30; a saturated hydrocarbon which was originally contained in the hydrocarbon stream 24; hydrogen fed to the first reaction zone 30 via stream 32; hydrogen which was originally contained in the hydrocarbon stream 24; or combinations thereof.

Within the second reaction zone 35, the reaction medium can comprise an unsaturated hydrocarbon which can be the product of a hydrogenation of the highly unsaturated hydrocarbon in the first reaction zone 30; an unsaturated hydrocarbon which can be the product of the hydrogenation of the highly unsaturated hydrocarbon in the second reaction zone 35; the unsaturated hydrocarbon which was originally contained in the hydrocarbon stream 24 and is not the product of hydrogenation in the first reaction zone 30; and a highly unsaturated hydrocarbon which was originally contained in the hydrocarbon stream 24 and is unreacted or unconverted in the second reaction zone 35. Additionally, the reaction medium within the second reaction zone 35 can comprise a saturated hydrocarbon which can be a side product of the hydrogenation reaction in the first reaction zone 30 and/or second reaction zone 35; the saturated hydrocarbon which was originally contained in the hydrocarbon stream 24; hydrogen fed to the first reaction zone 30 via stream 32 (and passed to the second reaction zone 35); hydrogen which was originally contained in the hydrocarbon stream 24; or combinations thereof.

The second effluent stream 36 can comprise an unsaturated hydrocarbon which can be the product of the hydrogenation of a highly unsaturated hydrocarbon in the first reaction zone 30; an unsaturated hydrocarbon which can be the product of the hydrogenation of the highly unsaturated hydrocarbon in the second reaction zone 35; the unsaturated hydrocarbon which was originally contained in the hydrocarbon stream 24 and is not the product of hydrogenation in the first reaction zone 30 or second reaction zone 35; and a highly unsaturated hydrocarbon which was originally contained in the hydrocarbon stream 24 and is unreacted or unconverted in the second reaction zone 35. Additionally, the second effluent stream 36 can comprise a saturated hydrocarbon which can be a side product of the hydrogenation reaction in the first reaction zone 30 and/or second reaction zone 35; the saturated hydrocarbon which was originally contained in the hydrocarbon stream 24; hydrogen fed to the first reaction zone 30 via stream 32 (and passed to the second reaction zone 35); hydrogen; carbon monoxide which was originally contained in the hydrocarbon stream 24; or combinations thereof.

The saturated hydrocarbon stream 42 can comprise a saturated hydrocarbon such as ethane and/or methane which can be recovered by the fractionation zone 40. The unsaturated hydrocarbon stream 44 can comprise an unsaturated hydrocarbon such as ethylene which can be recovered by the fractionation zone 40.

In embodiments where the highly unsaturated hydrocarbon fed to the first reaction zone 30 comprises acetylene, the mole ratio of hydrogen to the acetylene being fed to the first reaction zone 30 can be in the range of from about 10 to about 3000; alternatively, from about 10 to about 2000; alternatively, from about 10 to about 1500. In embodiments where the highly unsaturated hydrocarbon fed to the first reaction zone 30 is in a demethanizer configuration the mole ratio of hydrogen to the acetylene being fed to the first reaction zone 30 can be in the range of from about 0.1 to about 10; alternatively, from about 0.2 to about 5; alternatively, from about 0.5 to about 3.

In embodiments, the first reaction zone 30 can comprise a first hydrogenation catalyst, and the second reaction zone 35 can comprise a second hydrogenation catalyst. Generally, at least one of the first hydrogenation catalyst and the second hydrogenation catalyst can comprise a hydrogenation catalyst, and optionally, an organophosphorus compound. Suitable hydrogenation catalysts are described in detail herein. The first hydrogenation catalyst and the second hydrogenation catalyst can comprise the same or different catalyst compositions which have the selectivity to the unsaturated hydrocarbon as disclosed herein. Generally, the first hydrogenation catalyst and the second hydrogenation catalyst have a selectivity to an unsaturated hydrocarbon (e.g., ethylene) of about 90 mol % or greater based on moles of the highly unsaturated hydrocarbon (e.g., acetylene) which are converted in the respective reaction zone. In embodiments, the first hydrogenation catalyst, the second hydrogenation catalyst, or both comprise a high selectivity (e.g., about 90 mol % or greater based on moles of the highly unsaturated hydrocarbon converted) from start of run to end of run for a embodiments of the processes disclosed herein.

The first reaction zone 30, second reaction zone 35, or both can operate at conditions (e.g., gas phase, liquid phase, or both) effective to hydrogenate a highly unsaturated hydrocarbon to one or more compounds comprising an unsaturated hydrocarbon and optionally a saturated hydrocarbon in the presence of the first and second hydrogenation catalyst (and/or upon contacting the hydrocarbon stream 24 with the first and second hydrogenation catalyst) and in the presence of hydrogen.

In embodiments, a conversion of a highly unsaturated hydrocarbon to one or more compounds comprising an unsaturated hydrocarbon and optionally a saturated hydrocarbon after hydrogenation of the highly unsaturated hydrocarbon in the first reaction zone 30 is about 90 mol %, 91 mol %, 92 mol %, 93 mol %, 94 mol %, 95 mol %, 96 mol %, 97 mol %, 98 mol %, 99 mol % or greater. In embodiments, a total conversion of the highly unsaturated hydrocarbon to the unsaturated hydrocarbon and optionally the saturated hydrocarbon after hydrogenation of the highly unsaturated hydrocarbon in the first reaction zone 30 and after hydrogenation of the unreacted or the unconverted highly unsaturated hydrocarbon (e.g., unreacted or unconverted in the first reaction zone 30) in the second reaction zone 35 is about 99 mol %, 99.1 mol %, 99.2 mol %, 99.3 mol %, 99.4 mol %, 99.5 mol %, 99.6 mol %, 99.7 mol %, 99.8 mol %, 99.9 mol %, 99.99 mol %, 99.999 mol %, 99.9999 mol % or greater. In embodiments, a conversion of the highly unsaturated hydrocarbon to one or more compounds comprising the unsaturated hydrocarbon and optionally the saturated hydrocarbon after hydrogenation of the highly unsaturated hydrocarbon in the presence of the first hydrogenation catalyst is about 90 mol %, 91 mol %, 92 mol %, 93 mol %, 94 mol %, 95 mol %, 96 mol %, 97 mol %, 98 mol %, 99 mol % or greater. In embodiments, a total conversion of the highly unsaturated hydrocarbon to the unsaturated hydrocarbon and optionally the saturated hydrocarbon after hydrogenation of the highly unsaturated hydrocarbon in the presence of a first hydrogenation catalyst and after hydrogenation of the unreacted or the unconverted highly unsaturated hydrocarbon (e.g., unreacted or unconverted by the first hydrogenation catalyst) in the presence of the second hydrogenation catalyst is about 99 mol %, 99.1 mol %, 99.2 mol %, 99.3 mol %, 99.4 mol %, 99.5 mol %, 99.6 mol %, 99.7 mol %, 99.8 mol %, 99.9 mol %, 99.99 mol %, 99.999 mol %, 99.9999 mol % or greater.

The conversions disclosed herein are attained in combination with the hydrogenation selectivity of the catalysts disclosed herein.

The first hydrogenation catalyst and the second hydrogenation catalyst can be collectively referred to as the hydrogenation catalyst. Embodiments of the hydrogenation catalysts described herein can generally be used for selectively hydrogenating a highly unsaturated hydrocarbon to an unsaturated hydrocarbon. For example, the hydrogenation catalyst can be contacted with at least a portion of the highly unsaturated hydrocarbon in the presence of hydrogen in at least one of the first reaction zone 30 and the second reaction zone 35.

In embodiments, the hydrogenation catalyst can comprise any composition used for the hydrogenation of a highly unsaturated hydrocarbon to an unsaturated hydrocarbon which has a selectivity or hydrogenation selectivity for the conversion of a highly saturated hydrocarbon (e.g., acetylene) to an unsaturated hydrocarbon (e.g., ethylene) of about 90 mol %, 91 mol %, 92 mol %, 93 mol %, 94 mol %, 95 mol %, 96 mol %, 97 mol %, 98 mol %, 99 mol % or greater based on the moles of the highly unsaturated hydrocarbon which are converted to the unsaturated hydrocarbon, while operating at the conversions disclosed herein. Herein “selectivity” or “hydrogenation selectivity” generally refers to the amount of the converted highly unsaturated hydrocarbon (e.g., acetylene) which is converted to the unsaturated hydrocarbon (e.g., ethylene). For example, at a total conversion of 99 mol %, 99 moles of the highly unsaturated hydrocarbon would be converted to a product made of compounds such as the unsaturated hydrocarbon and saturated hydrocarbon, while one mole of the highly unsaturated hydrocarbon remains unconverted or unreacted. A selectivity of 90.9 mol % to the unsaturated hydrocarbon when total conversion is at 99 mol % can indicate that, for example, of the 99 moles of the highly unsaturated hydrocarbon which were converted, 90 moles of the highly unsaturated hydrocarbon were converted to the unsaturated hydrocarbon while 9 moles of the highly unsaturated hydrocarbon were converted to other compounds such as a saturated hydrocarbon or other side products of the hydrogenation reaction.

In embodiments, the selectivity can be defined as:

$$S = 100 \times ( \frac{UH ( p ) - UH ( f )}{HUH ( f ) - HUH ( p )} )$$

where S is selectivity in mol %, UH(p) is moles of the unsaturated hydrocarbon in the product, UH(f) is moles of the unsaturated hydrocarbon in the hydrocarbon stream, HUH(f) is the moles of highly unsaturated hydrocarbon in the hydrocarbon stream, and HUH(p) is the moles of the highly unsaturated hydrocarbon in the product.

In embodiments, the hydrogenation catalyst can comprise an inorganic support and palladium. In additional embodiments, the hydrogenation catalyst can further comprise an organophosphorus compound (e.g., impregnated in or on the inorganic support thereof).

In an embodiment, the inorganic support can comprise aluminas, silicas, titanias, zirconias, aluminosilicates (e.g., clays, ceramics, and/or zeolites), spinels (e.g., zinc aluminate, zinc titanate, and/or magnesium aluminate), or combinations thereof. In an embodiment, the support can comprise an alumina support. In some embodiments, the alumina support can comprise an alpha (α)-alumina support or a chloride-treated alpha alumina support.

The inorganic support can have a surface area of from about 2 to about 100 square meters per gram (m²/g); alternatively, of from about 2 m²/g to about 75 m²/g; alternatively, of from about 3 m²/g to about 50 m²/g; alternatively, of from about 4 m²/g to about 25 m²/g; alternatively, of from about 5 m²/g to about 10 m²/g. The surface area of the support can be determined using any suitable method. An example of a suitable method includes the Brunauer, Emmett, and Teller (“BET”) method, which measures the quantity of nitrogen adsorbed on the support. Alternatively, the surface area of the support can be measured by a mercury intrusion method such as is described in ASTM UOP 578-02, entitled “Automated Pore Volume and Pore Size Distribution of Porous Substances by MERCURY Porosimetry,” which is incorporated herein by reference in its entirety.

Particles of the inorganic support generally have an average diameter of from about 1 mm to about 10 mm; alternatively, from about 1 mm to about 6 mm; alternatively, from about 2 mm to about 6 mm; alternatively, from about 3 mm to about 5 mm. The inorganic support can have any suitable shape, including round or spherical (e.g., spheres), ellipsoidal, pellets, cylinders, granules (e.g., regular and/or irregular), trilobe, quadrilobe, rings, wagonwheel, and monoliths. Methods for shaping particles include, for example, extrusion, spray drying, pelletizing, marumerizing, agglomeration, oil drop, and the like. In an embodiment, the shape of the inorganic support can be cylindrical. In an alternative embodiment, the shape of the inorganic support can be spherical.

In an embodiment, the inorganic support can be present in an amount such that it comprises the balance of the hydrogenation catalyst when all other components are accounted for.

In an embodiment, the hydrogenation catalyst can comprise palladium. The palladium can be added to the inorganic support by contacting the inorganic support with a palladium-containing compound to form a palladium supported catalyst as will be described in more detail later herein. Examples of suitable palladium-containing compounds include without limitation palladium chloride, palladium nitrate, ammonium hexachloropalladate, ammonium tetrachlopalladate, palladium acetate, palladium bromide, palladium iodide, tetraamminepalladium nitrate, or combinations thereof. In an embodiment, the palladium-containing compound is a component of an aqueous solution. In an embodiment, the palladium-containing compound can be a component of an acidic solution, e.g., an aqueous solution comprising a mineral acid. An example of palladium-containing solution suitable for use in this disclosure includes without limitation a solution comprising palladium metal.

In an embodiment, the hydrogenation catalyst can be prepared using a palladium-containing compound in an amount of from about 0.005 wt. % to about 5 wt. % based on the total weight of the hydrogenation catalyst; alternatively, from about 0.01 wt. % to about 3 wt. %; alternatively, from about 0.02 wt. % to about 1 wt. %; alternatively, from about 0.02 wt. % to about 0.04 wt. %; alternatively, from about 0.02 wt. % to about 0.1 wt. %. The amount of palladium incorporated into the hydrogenation catalyst can be in the range described herein for the amount of palladium-containing compound used to prepare the hydrogenation catalyst.

In an embodiment, the hydrogenation catalyst can further comprise an organophosphorus compound. In an embodiment, the organophosphorus compound can be represented by the general formula of (R)ₓ(OR′)_{y}P═O; wherein x and y are integers ranging from 0 to 3 and x plus y equals 3; wherein each R can be hydrogen, a hydrocarbyl group, or combinations thereof; and wherein each R′ can a hydrocarbyl group. In some embodiments, the organophosphorus compound can include compounds such as phosphine oxides, phosphinates, phosphonates, phosphates, or combinations of any of the foregoing. For purposes of this application, the term “hydrocarbyl(s)” or “hydrocarbyl group(s)” is used herein in accordance with the definition specified by IUPAC: as a univalent group or groups derived by the removal of one hydrogen atom from a carbon atom of a “hydrocarbon.” A hydrocarbyl group can be an aliphatic, inclusive of acyclic and cyclic groups. A hydrocarbyl group can include rings, ring systems, aromatic rings, and aromatic ring systems. Hydrocarbyl groups can include, by way of example, aryl, alkyl, cycloalkyl, and combinations of these groups, among others. Hydrocarbyl groups can be linear or branched unless otherwise specified. For the purposes of this application, the terms “alkyl,” or “cycloalkyl” refers to a univalent group derived by removal of a hydrogen atom from any carbon atom of an alkane. For the purposes of this application, the terms “aryl,” or “arylene” refers to a univalent group derived by removal of a hydrogen atom from any carbon atom of an aryl ring.

In an embodiment, the hydrocarbyl group can have from 1 to 30 carbon atoms; alternatively, from 2 to 20 carbon atoms; alternatively, from 3 to 15 carbon atoms. In other embodiments, the hydrocarbyl group can have from about 6 to about 30 carbon atoms; alternatively, from about 6 to about 20 carbon atoms; alternatively, from about 6 to about 15 carbon atoms.

Generally, the alkyl group for any feature which calls for an alkyl group described herein can be a methyl, ethyl, n-propyl (1-propyl), isopropyl (2-propyl), n-butyl (1-butyl), sec-butyl (2-butyl), isobutyl (2-methyl-1-propyl), tert-butyl (2-methyl-2-propyl), n-pentyl (1-pentyl), 2-pentyl, 3-pentyl, 2-methyl-1-butyl, tert-pentyl (2-methyl-2-butyl), 3-methyl-1-butyl, 3-methyl-2-butyl, neo-pentyl (2,2-dimethyl-1-propyl), n-hexyl (1-hexyl) group. Persons having ordinary skill in the art with the aids of this disclosure will readily recognize which alkyl group represents primary, secondary, or tertiary alkyl groups.

Organophosphorus compounds described herein are not considered to encompass elemental phosphorus, or inorganic phosphorus compounds, except that which can be produced during the preparation of the hydrogenation catalyst described herein. Inorganic phosphorus compounds encompass monobasic, dibasic, and tribasic phosphates such as tribasic potassium phosphate (K₃PO₄), tribasic sodium phosphate (Na₃PO₄), dibasic potassium phosphate (K₂HPO₄), dibasic sodium phosphate (Na₂HPO₄), monobasic potassium phosphate (KH₂PO₄), monobasic sodium phosphate (NaH₂PO₄). Inorganic phosphorus compounds also encompass the corresponding phosphorus acid of above mentioned salts. Inorganic phosphorus compounds also encompass anionic inorganic phosphorus compounds containing pentavalent phosphorus, and halogens. Examples of anionic inorganic phosphorus compounds include sodium and potassium hexafluorophosphate.

An organophosphorus compound suitable for use in this disclosure can be further characterized by a low boiling point wherein a low boiling point refers to a boiling point of about 100° C. Alternatively, an organophosphorus compound suitable for use in this disclosure can be further characterized by a high boiling point wherein a high boiling point refers to a boiling point of equal to or greater than about 100° C.

In an embodiment, the organophosphorus compound can comprise a phosphine oxide which can be represented by the general formula of (R)₃P═O; wherein each R can be hydrogen, a hydrocarbyl group, or combinations thereof. Examples of phosphine oxides suitable for use in this disclosure include without limitation butyldiethylphosphine oxide, butyldimethylphosphine oxide, butyldiphenylphosphine oxide, butyldipropylphosphine oxide, decyldiethylphosphine oxide, decyldimethylphosphine oxide, decyldiphenylphosphine oxide, dibutyl(2-methylphenyl)-phosphine oxide, diethyl(3-methylphenyl)-phosphine oxide, ethyldioctylphosphine oxide, ethyldibutylphosphine oxide, ethyldimethylphosphine oxide, ethyldiphenylphosphine oxide, ethyldipropylphosphine oxide, heptyldibutylphosphine oxide, heptyldiethylphosphine oxide, heptyldimethyl phosphine oxide, heptyldipentylphosphine oxide, heptyldiphenylphosphine oxide, hexyldibutylphosphine oxide, hexyldiethylphosphine oxide, hexyldimethyl phosphine oxide, hexyldipentylphosphine oxide, hexyldiphenylphosphine oxide, methylbis(4-methylphenyl)-phosphine oxide, methyldibutylphosphine oxide, methyldidecylphosphine oxide, methyldiethylphosphine oxide, methyldiphenylphosphine oxide, methyldipropylphosphine oxide, octyldimethylphosphine oxide, octyldiphenylphosphine oxide, pentyldibutylphosphine oxide, pentyldiethylphosphine oxide, pentyldimethylphosphine oxide, pentyldiphenylphosphine oxide, phenyldibutylphosphine oxide, phenyldiethylphosphine oxide, phenyldimethylphosphine oxide, phenyldipropylphosphine oxide, propyldibutylphosphine oxide, propyldimethylphosphine oxide, propyldiphenylphosphine oxide, tris(2,6-dimethylphenyl)-phosphine oxide, tris(2-methylphenyl)-phosphine oxide, tris(4-methylphenyl)-phosphine oxide, tris[4-(1,1-dimethylethyl)phenyl]-phosphine oxide, (1-methylethyl)diphenyl-phosphine oxide, 4-(diphenylmethyl)phenyl]diphenyl-phosphine oxide, bis(2-methylphenyl)(2-methylpropyl)-phosphine oxide, or combinations thereof. In some embodiments, the phosphine oxides suitable for use in this disclosure include without limitation tributylphosphine oxide, triethylphosphine oxide, triheptylphosphine oxide, trimethylphosphine oxide, trioctylphosphine oxide, tripentylphosphine oxide, tripropylphosphine oxide, triphenylphosphine oxide, or combinations thereof.

In an embodiment, the organophosphorus compound can comprise an organic phosphate which can be represented by the general formula of (OR′)₃P═O; wherein each R′ can a hydrocarbyl group. Examples of phosphates suitable for use in this disclosure include without limitation (1-methylethyl)diphenyl phosphate, 2-ethylphenyldiphenyl phosphate, 4-(diphenylmethyl)phenyl]diphenyl phosphate, bis(2-methylphenyl)(2-methylpropyl) phosphate, butyldiethylphosphate, butyldimethylphosphate, butyldiphenylphosphate, butyldipropylphosphate, crecyldiphenylphosphate, decyldiethylphosphate, decyldimethylphosphate, decyldiphenylphosphate, dibutyl(2-methylphenyl) phosphate, diethyl(3-methylphenyl) phosphate, ethyldibutylphosphate, ethyldimethylphosphate, ethyldioctylphosphate, ethyldiphenylphosphate, ethyldipropylphosphate, heptyldibutylphosphate, heptyldiethylphosphate, heptyldimethyl phosphate, heptyldipentylphosphate, heptyldiphenylphosphate, hexyldibutylphosphate, hexyldiethylphosphate, hexyldimethyl phosphate, hexyldipentylphosphate, hexyldiphenylphosphate, methylbis(4-methylphenyl) phosphate, methyldibutylphosphate, methyldidecylphosphate, methyldiethylphosphate, methyldiphenylphosphate, methyldipropylphosphate, octyldimethylphosphate, octyldiphenylphosphate, pentyldibutylphosphate, pentyldiethylphosphate, pentyldimethylphosphate, pentyldiphenylphosphate, phenyldibutylphosphate, phenyldiethylphosphate, phenyldimethylphosphate, phenyldipropylphosphate, prop yldibutylphosphate, propyldimethylphosphate, propyldiphenylphosphate, tri(2,3-dichloropropyl) phosphate, tri(2,6-dimethylphenyl) phosphate, tri(2-chloroethyl) phosphate, tri(nonylphenyl) phosphate, tris(2,6-dimethylphenyl) phosphate, tris(2-methylphenyl) phosphate, tris(4-methylphenyl) phosphate, tris[4-(1,1-dimethylethyl)phenyl] phosphate, or combinations thereof. In some embodiments, the phosphates suitable for use in this disclosure include without limitation tributylphosphate, tricresyl phosphate, tricyclohexyl phosphate, tridecylphosphate, triethylphosphate, triheptylphosphate, triisopropyl phosphate, trimethylphosphate, trioctadecyl phosphate, trioctylphosphate, tripentylphosphate, triphenylphosphate, tripropylphosphate, trixylylphosphate, or combinations thereof.

In an embodiment, the organophosphorus compound can comprise a phosphinate, which can be represented by the general formula of (R)₂(OR′)P═O; wherein each R can be hydrogen, a hydrocarbyl group, or combinations thereof; and wherein each R′ can a hydrocarbyl group. Examples of phosphinates suitable for use in this disclosure include without limitation butyl butylphosphinate, butyl dibutylphosphinate, butyl diethylphosphinate, butyl diphenylphosphinate, butyl dipropylphosphinate, butyl ethylphosphinate, butyl heptylphosphinate, butyl hexylphosphinate, butyl pentylphosphinate, butyl phenylphosphinate, butyl propylphosphinate, decyl pentylphosphinate, butyl butylpentylphosphinate, ethyl butylphosphinate, ethyl decylphosphinate, ethyl dibutylphosphinate, ethyl diethylphosphinate, ethyl dimethylphosphinate, ethyl diphenylphosphinate, ethyl dipropylphosphinate, ethyl ethylphosphinate, ethyl heptylphosphinate, ethyl hexylphosphinate, ethyl octylphosphinate, ethyl pentylphosphinate, ethyl phenylphosphinate, ethyl propylphosphinate, heptyl dibutylphosphinates, heptyl pentylphosphinate, heptylphosphinate, hexyl dibutylphosphinate, hexyl pentylphosphinate, isopropyl diphenylphosphinate, methyl butylphosphinate, methyl decylphosphinate, methyl dibutylphosphinate, methyl diethylphosphinate, methyl dimethylphosphinate, methyl diphenylphosphinates, methyl dipropylphosphinate, methyl ethylphosphinate, methyl heptylphosphinate, methyl hexylphosphinate, methyl octylphosphinate, methyl pentylphosphinate, methyl phenylphosphinate, methyl propylphosphinate, octyl pentylphosphinate, octylphosphinate, pentyl dibutylphosphinate, pentylphosphinate, phenyl butylphosphinate, phenyl decylphosphinate, phenyl dibutylphosphinate, phenyl diethylphosphinate, phenyl diethylphosphinate, phenyl dimethylphosphinate, phenyl diphenylphosphinate, phenyl diphenylphosphinate, phenyl dipropylphosphinate, phenyl ethylphosphinate, phenyl heptylphosphinate, phenyl hexylphosphinate, phenyl octylphosphinate, phenyl pentylphosphinate, phenyl pentylphosphinate, phenyl phenylphosphinate, phenyl propylphosphinate, phenylphosphinate, propyl diphenylphosphinate, or combinations thereof.

In an embodiment, the organophosphorus compound can comprise a phosphonate, which can be represented by the general formula of (R)(OR′)₂P═O; wherein each R can be hydrogen, a hydrocarbyl group, or combinations thereof; and wherein each R′ can a hydrocarbyl group. Examples of phosphonates suitable for use in this disclosure include without limitation (1-methylethyl)diphenyl phosphonate, 2-ethylphenyldiphenyl phosphonate, 4-(diphenylmethyl)phenyl]diphenyl phosphonate, bis(2-methylphenyl) (2-methylpropyl) phosphonate, butyldiethylphosphonate, butyldimethylphosphonate, butyldiphenylphosphonate, butyldipropylphosphonate, crecyldiphenylphosphonate, decyldiethylphosphonate, decyldimethylphosphonate, decyldiphenylphosphonate, dibutyl(2-methylphenyl) phosphonate, diethyl(3-methylphenyl) phosphonate, ethyldibutylphosphonate, ethyldimethylphosphonate, ethyldioctylphosphonate, ethyldiphenylphosphonate, ethyldipropylphosphonate, heptyldibutylphosphonate, heptyldiethylphosphonate, heptyldimethyl phosphonate, heptyldipentylphosphonate, heptyldiphenylphosphonate, hexyldibutylphosphonate, hexyldiethylphosphonate, hexyldimethyl phosphonate, hexyldipentylphosphonate, hexyldiphenylphosphonate, methylbis(4-methylphenyl) phosphonate, methyldibutylphosphonate, methyldidecylphosphonate, methyldiethylphosphonate, methyldiphenylphosphonate, methyldipropylphosphonate, octyldimethylphosphonate, octyldiphenylphosphonate, pentyldibutylphosphonate, pentyldiethylphosphonate, pentyldimethylphosphonate, pentyldiphenylphosphonate, phenyldibutylphosphonate, phenyldiethylphosphonate, phenyldimethylphosphonate, phenyldipropylphosphonate, propyldibutylphosphonate, propyldimethylphosphonate, propyldiphenylphosphonate, tri(2,3-dichloropropyl) phosphonate, tri(2,6-dimethylphenyl) phosphonate, tri(2-chloroethyl) phosphonate, tri(nonylphenyl) phosphonate, tris(2,6-dimethylphenyl) phosphonate, tris(2-methylphenyl) phosphonate, tris(4-methylphenyl) phosphonate, tris[4-(1, 1-dimethylethyl)phenyl] phosphonate, or combinations thereof. In some embodiments, the phosphonates suitable for use in this disclosure include without limitation tributylphosphonate, tricresyl phosphonate, tricyclohexyl phosphonate, tridecylphosphonate, triethylphosphonate, triheptylphosphonate, triisopropyl phosphonate, trimethylphosphonate, trioctadecyl phosphonate, trioctylphosphonate, tripentylphosphonate, triphenylphosphonate, tripropylphosphonate, trixylylphosphonate, or combinations thereof.

In an embodiment, the hydrogenation catalyst can comprise a precursor to the organophosphorus compound. The organophosphorus compound precursor can comprise any material which can be converted to the organophosphorus compound which activates the hydrogenation catalyst under the conditions to which the hydrogenation catalyst is exposed and that is compatible with the other components of the hydrogenation catalyst. In an embodiment, the organophosphorus compound precursor can be represented by the general formula of (R)ₓ(OR′)_{y}P; wherein x and y are integers ranging from 0 to 3 and x plus y equals 3; wherein each R can be hydrogen, a hydrocarbyl group, or combinations thereof; and wherein each R′ can a hydrocarbyl group. The organophosphorus compound precursor can include without limitation phosphines, phosphites, phosphinites, phosphonites, or combinations thereof. In an embodiment, the organophosphorus compound precursor can comprise a phosphine that can form a phosphine oxide when exposed to an oxidizing agent and/or temperatures greater than about 20° C. In an embodiment, the organophosphorus compound precursor can comprise a phosphite that can form a phosphate when exposed to an oxidizing agent and/or temperatures greater than about 20° C. In an embodiment, the organophosphorus compound precursor can comprise a phosphinite that can form a phosphinate when exposed to oxidizing agent and/or temperatures greater than about 20° C. In an embodiment, the organophosphorus compound precursor can comprise a phosphonite that can form a phosphonate when exposed to air and/or temperatures greater than about 20° C.

In an embodiment, the organophosphorus compound can comprise phosphines, which can be represented by the general formula of (R)₃P; wherein each R can be hydrogen, a hydrocarbyl group, or combinations thereof. Examples of phosphines suitable for use as phosphine oxide precursors in this disclosure include without limitation (1-methylethyl)diphenylphosphine, 2-ethylphenyldiphenyl phosphine, 4-(diphenylmethyl)phenyl]diphenylphosphine, bis(2-methylphenyl) (2-methylpropyl) phosphine, butyldiethylphosphine, butyldimethylphosphine, butyldiphenylphosphine, butyldipropylphosphine, crecyldiphenylphosphine, cyclohexyldiphenylphosphine, decyldiethylphosphine, decyldimethylphosphine, decyldiphenylphosphine, dibutyl(2-methylphenyl) phosphine, dicyclohexylphenylphosphine, diethyl(3-methylphenyl)phosphine, ethyldibutylphosphine, ethyldimethylphosphine, ethyldioctylphosphine, ethyldiphenylphosphine, ethyldipropylphosphine, heptyldibutylphosphine, heptyldiethylphosphine, heptyldimethyl phosphine, heptyldipentylphosphine, heptyldiphenylphosphine, hexyldibutylphosphine, hexyldiethylphosphine, hexyldimethyl phosphine, hexyldipentylphosphine, hexyldiphenylphosphine, methylbis(4-methylphenyl) phosphine, methyldibutylphosphine, methyldidecylphosphine, methyldiethylphosphine, methyldiphenylphosphine, methyldipropylphosphine, octyldimethylphosphine, octyldiphenylphosphine, pentyldibutylphosphine, pentyldiethylphosphine, pentyldimethylphosphine, pentyldiphenylphosphine, phenyldibutylphosphine, phenyldiethylphosphine, phenyldimethylphosphine, phenyldipropylphosphine, propyldibutylphosphine, propyldimethylphosphine, propyldiphenylphosphine, tri(2,3-dichloropropyl) phosphine, tri(2,6-dimethylphenyl) phosphine, tri(2-chloroethyl) phosphine, tri(nonylphenyl) phosphine, tris(2,6-dimethylphenyl) phosphine, tris(2-methylphenyl) phosphine, tris(4-methylphenyl) phosphine, tris(methoxyphenyl)phosphine, tris[4-(1, 1-dimethylethyl)phenyl] phosphine, or combinations thereof. In some embodiments, the phosphines suitable for use in this disclosure include without limitation tributylphosphine, tricresyl phosphine, tricyclohexyl phosphine, tridecylphosphine, triethylphosphine, triheptylphosphine, triisopropylphosphine, trimethylphosphine, trioctadecyl phosphine, trioctylphosphine, tripentylphosphine, triphenylphosphine, tripropylphosphine, tri-t-butylphosphine, tritolylphosphine, trixylylphosphine, or combinations thereof.

In an embodiment, the organophosphorus compound can comprise phosphites, which can be represented by the general formula of (OR′)₃P; wherein each R′ can a hydrocarbyl group. Examples of phosphites suitable for use as phosphate precursors in this disclosure include without limitation (1-methylethyl)diphenylphosphite, 2-ethylphenyldiphenyl phosphite, 4-(diphenylmethyl)phenyl]diphenylphosphite, bis(2-methylphenyl)(2-methylpropyl) phosphite, butyldiethylphosphite, butyldimethylphosphite, butyldiphenylphosphite, butyldipropylphosphite, crecyldiphenylphosphite, cyclohexyldiphenylphosphite, decyldiethylphosphite, decyldimethylphosphite, decyldiphenylphosphite, dibutyl(2-methylphenyl) phosphite, dicyclohexylphenylphosphite, diethyl(3-methylphenyl)phosphite, ethyldibutylphosphite, ethyldimethylphosphite, ethyldioctylphosphite, ethyldiphenylphosphite, ethyldipropylphosphite, heptyldibutylphosphite, heptyldiethylphosphite, heptyldimethyl phosphite, heptyldipentylphosphite, heptyldiphenylphosphite, hexyldibutylphosphite, hexyldiethylphosphite, hexyldimethyl phosphite, hexyldipentylphosphite, hexyldiphenylphosphite, methylbis(4-methylphenyl) phosphite, methyldibutylphosphite, methyldidecylphosphite, methyldiethylphosphite, methyldiphenylphosphite, methyldipropylphosphite, octyldimethylphosphite, octyldiphenylphosphite, pentyldibutylphosphite, pentyldiethylphosphite, pentyldimethylphosphite, pentyldiphenylphosphite, phenyldibutylphosphite, phenyldiethylphosphite, phenyldimethylphosphite, phenyldipropylphosphite, propyldibutylphosphite, propyldimethylphosphite, propyldiphenylphosphite, tri(2-chloroethyl) phosphite, tri(nonylphenyl) phosphite, tris(2,3-dichloropropyl) phosphite, tris(2,6-dimethylphenyl) phosphite, tris(2-methylphenyl) phosphite, tris(4-methylphenyl) phosphite, tris(methoxyphenyl)phosphite, tris[4-(1,1-dimethylethyl)phenyl] phosphite, tri-t-butylphosphite, or combinations thereof. In some embodiments, the phosphites suitable for use in this disclosure include without limitation tributylphosphite, tricresyl phosphite, tricyclohexyl phosphite, tridecylphosphite, triethylphosphite, triheptylphosphite, triisopropylphosphite, trimethylphosphite, trioctadecyl phosphite, trioctylphosphite, tripentylphosphite, triphenylphosphite, tripropylphosphite, tritolylphosphite, trixylylphosphite, or combinations thereof.

In an embodiment, the organophosphorus compound can comprise phosphinites, which can be represented by the general formula of (R)₂(OR′)₁P; wherein each R can be hydrogen, a hydrocarbyl group, or combinations thereof; and wherein each R′ can a hydrocarbyl group. Examples of phosphinites suitable for use as phosphate precursors in this disclosure include without limitation (1-methylethyl)diphenylphosphinite, 2-ethylphenyldiphenyl phosphinite, 4-(diphenylmethyl)phenyl]diphenylphosphinite, bis(2-methylphenyl)(2-methylpropyl) phosphinite, butyldiethylphosphinite, butyldimethylphosphinite, butyldiphenylphosphinite, butyldipropylphosphinite, crecyldiphenylphosphinite, cyclohexyldiphenylphosphinite, decyldiethylphosphinite, decyldimethylphosphinite, decyldiphenylphosphinite, dibutyl(2-methylphenyl) phosphinite, dicyclohexylphenylphosphinite, diethyl(3-methylphenyl)phosphinite, ethyldibutylphosphinite, ethyldimethylphosphinite, ethyldioctylphosphinite, ethyldiphenylphosphinite, ethyldipropylphosphinite, heptyldibutylphosphinite, heptyldiethylphosphinite, heptyldimethyl phosphinite, heptyldipentylphosphinite, heptyldiphenylphosphinite, hexyldibutylphosphinite, hexyldiethylphosphinite, hexyldimethyl phosphinite, hexyldipentylphosphinite, hexyldiphenylphosphinite, methylbis(4-methylphenyl) phosphinite, methyldibutylphosphinite, methyldidecylphosphinite, methyldiethylphosphinite, methyldiphenylphosphinite, methyldipropylphosphinite, octyldimethylphosphinite, octyldiphenylphosphinite, pentyldibutylphosphinite, pentyldiethylphosphinite, pentyldimethylphosphinite, pentyldiphenylphosphinite, phenyldibutylphosphinite, phenyldiethylphosphinite, phenyldimethylphosphinite, phenyldipropylphosphinite, propyldibutylphosphinite, propyldimethylphosphinite, propyldiphenylphosphinite, tri(2-chloroethyl) phosphinite, tri(nonylphenyl) phosphinite, tris(2,3-dichloropropyl) phosphinite, tris(2,6-dimethylphenyl) phosphinite, tris(2-methylphenyl) phosphinite, tris(4-methylphenyl) phosphinite, tris(methoxyphenyl)phosphinite, tris[4-(1,1-dimethylethyl)phenyl] phosphinite, tri-t-butylphosphinite, or combinations thereof. In some embodiments, the phosphinites suitable for use in this disclosure include without limitation tributylphosphinite, tricresyl phosphinite, tricyclohexyl phosphinite, tridecylphosphinite, triethylphosphinite, triheptylphosphinite, triisopropylphosphinite, trimethylphosphinite, trioctadecyl phosphinite, trioctylphosphinite, tripentylphosphinite, triphenylphosphinite, tripropylphosphinite, tritolylphosphinite, trixylylphosphinite, or combinations thereof.

In an embodiment, the organophosphorus compound can comprise phosphonites, which can be represented by the general formula of (R)₁(OR′)₂P; wherein each R can be hydrogen, a hydrocarbyl group, or combinations thereof; and wherein each R′ can a hydrocarbyl group. Examples of phosphonites suitable for use as phosphate precursors in this disclosure include without limitation (1-methylethyl)diphenylphosphonite, 2-ethylphenyldiphenyl phosphonite, 4-(diphenylmethyl)phenyl]diphenylphosphonite, bis(2-methylphenyl)(2-methylpropyl) phosphonite, butyldiethylphosphonite, butyldimethylphosphonite, butyldiphenylphosphonite, butyldipropylphosphonite, crecyldiphenylphosphonite, cyclohexyldiphenylphosphonite, decyldiethylphosphonite, decyldimethylphosphonite, decyldiphenylphosphonite, dibutyl(2-methylphenyl) phosphonite, dicyclohexylphenylphosphonite, diethyl(3-methylphenyl)phosphonite, ethyldibutylphosphonite, ethyldimethylphosphonite, ethyldioctylphosphonite, ethyldiphenylphosphonite, ethyldipropylphosphonite, heptyldibutylphosphonite, heptyldiethylphosphonite, heptyldimethyl phosphonite, heptyldipentylphosphonite, heptyldiphenylphosphonite, hexyldibutylphosphonite, hexyldiethylphosphonite, hexyldimethyl phosphonite, hexyldipentylphosphonite, hexyldiphenylphosphonite, methylbis(4-methylphenyl) phosphonite, methyldibutylphosphonite, methyldidecylphosphonite, methyldiethylphosphonite, methyldiphenylphosphonite, methyldipropylphosphonite, octyldimethylphosphonite, octyldiphenylphosphonite, pentyldibutylphosphonite, pentyldiethylphosphonite, pentyldimethylphosphonite, pentyldiphenylphosphonite, phenyldibutylphosphonite, phenyldiethylphosphonite, phenyldimethylphosphonite, phenyldipropylphosphonite, propyldibutylphosphonite, propyldimethylphosphonite, propyldiphenylphosphonite, tri(2-chloroethyl) phosphonite, tri(nonylphenyl) phosphonite, tris(2,3-dichloropropyl) phosphonite, tris(2,6-dimethylphenyl) phosphonite, tris(2-methylphenyl) phosphonite, tris(4-methylphenyl) phosphonite, tris(methoxyphenyl)phosphonite, tris[4-(1,1-dimethylethyl)phenyl] phosphonite, tri-t-butylphosphonite, or combinations thereof. In some embodiments, the phosphonites suitable for use in this disclosure include without limitation tributylphosphonite, tricresyl phosphonite, tricyclohexyl phosphonite, tridecylphosphonite, triethylphosphonite, triheptylphosphonite, triisopropylphosphonite, trimethylphosphonite, trioctadecyl phosphonite, trioctylphosphonite, tripentylphosphonite, triphenylphosphonite, tripropylphosphonite, tritolylphosphonite, trixylylphosphonite, or combinations thereof.

In an embodiment, the organophosphorus compound and/or organophosphorus compound precursor can be present in the mixture for the preparation of the hydrogenation catalyst in an amount of from about 0.005 wt. % to about 5 wt. % based on the weight of phosphorus to the total weight of the hydrogenation catalyst; alternatively, from about 0.01 wt. % to about 1 wt. %; alternatively, from about 0.01 wt. % to about 0.5 wt. %. The amount of organophosphorus compound and/or phosphorus incorporated into the hydrogenation catalyst can be in the range described herein for the amount of organophosphorus compound and/or precursor used to prepare the hydrogenation catalyst. Additionally or alternatively, the amount of hydrogenation catalyst can have about 300 ppmw phosphorous.

In an embodiment, the hydrogenation catalyst can further comprise one or more selectivity enhancers. Suitable selectivity enhancers include, but are not limited to, Group 1B metals, Group 1B metal compounds, silver compounds, fluorine, fluoride compounds, sulfur, sulfur compounds, alkali metals, alkali metal compounds, alkaline metals, alkaline metal compounds, iodine, iodide compounds, or combinations thereof. In an embodiment, the hydrogenation catalyst can comprise one or more selectivity enhancers which can be present in total in the mixture for preparation of the hydrogenation catalyst in an amount of from about 0.001 to about 10 wt. % based on the total weight of the hydrogenation catalyst; alternatively, from about 0.01 to about 5 wt. %; alternatively, from about 0.01 to about 2 wt. %. The amount of selectivity enhancer incorporated into the hydrogenation catalyst can be in the range described herein for the preparation of the hydrogenation catalyst.

In an embodiment, the selectivity enhancer can comprise silver (Ag), silver compounds, or combinations thereof. Examples of suitable silver compounds include without limitation silver nitrate, silver acetate, silver bromide, silver chloride, silver iodide, silver fluoride, or combinations thereof. In an embodiment, the selectivity enhancer comprises silver nitrate. The hydrogenation catalyst can be prepared using silver nitrate in an amount of from about 0.005 wt. % to about 5 wt. % silver based on the total weight of the hydrogenation catalyst; alternatively, from about 0.01 wt. % to about 1 wt. % silver; alternatively, from about 0.01 wt. % to about 0.5 wt. %. The amount of silver incorporated into the hydrogenation catalyst can be in the range described herein for the amount of silver nitrate used to prepare the hydrogenation catalyst.

In an embodiment, the selectivity enhancer can comprise alkali metals, alkali metal compounds, or combinations thereof. Examples of suitable alkali metal compounds include without limitation elemental alkali metal, alkali metal halides (e.g., alkali metal fluoride, alkali metal chloride, alkali metal bromide, alkali metal iodide), alkali metal oxides, alkali metal carbonate, alkali metal sulfate, alkali metal phosphate, alkali metal borate, or combinations thereof. In an embodiment, the selectivity enhancer comprises potassium fluoride (KF). In another embodiment, the hydrogenation catalyst can be prepared using an alkali metal compound in an amount of from about 0.01 wt. % to about 5 wt. % based on the total weight of the hydrogenation catalyst; alternatively, from about 0.05 wt. % to about 2 wt. %; alternatively, from about 0.05 wt. % to about 1 wt. %. The amount of alkali metal incorporated into the hydrogenation catalyst can be in the range described herein for the amount of alkali metal compound used to prepare the hydrogenation catalyst.

In an embodiment, a method of preparing a hydrogenation catalyst can initiate with the contacting of an inorganic support with a palladium-containing compound to form a supported palladium composition. The contacting can be carried out using any suitable technique. For example, the inorganic support can be contacted with the palladium-containing compound by incipient wetness impregnation of the support with a palladium-containing solution. In such embodiments, the resulting supported palladium composition can have greater than about 90 wt. %; alternatively, from about 92 wt. % to about 98 wt. %; alternatively, from about 94 wt. % to about 96 wt. % of the palladium concentrated near the periphery of the palladium supported composition, as to form a palladium skin.

The palladium skin can be any thickness as long as such thickness can promote the hydrogenation processes disclosed herein. Generally, the thickness of the palladium skin can be in the range of from about 1 micron to about 3000 microns; alternatively, from about 5 microns to about 2000 microns; alternatively, from about 10 microns to about 1000 microns; alternatively, from about 50 microns to about 500 microns. Examples of such methods are further described in more details in U.S. Pat. Nos. 4,404,124 and 4,484,015, each of which is incorporated by reference herein in its entirety.

Any suitable method can be used for determining the concentration of the palladium in the skin of the palladium supported composition and/or the thickness of the skin. For example, one method involves breaking open a representative sample of the palladium supported composition particles and treating the palladium supported composition particles with a dilute alcoholic solution of N,N-dimethyl-para-nitrosoaniline. The treating solution reacts with the palladium to give a red color that can be used to evaluate the distribution of the palladium. Yet another technique for measuring the concentration of the palladium in the skin of the palladium supported composition involves breaking open a representative sample of catalyst particles, followed by treating the particles with a reducing agent such as hydrogen to change the color of the skin and thereby evaluate the distribution of the palladium. Alternatively, the palladium skin thickness can be determined using the electron microprobe method.

The supported palladium composition formed by contacting the inorganic support with the palladium-containing solution optionally can be dried at a temperature of from about 15° C. to about 150° C.; alternatively, from about 30° C. to about 140° C.; alternatively, from about 60° C. to about 130° C.; and for a period of from about 0.1 hour to about 100 hours; alternatively, from about 2 hours to about 200 hours; alternatively, from about 0.3 hour to about 10 hours. Alternatively, the palladium supported composition can be calcined. This calcining step can be carried out at temperatures up to about 850° C.; alternatively, of from about 150° C. to about 750° C.; alternatively, from about 150° C. to about 700° C.; alternatively, from about 150° C. to about 680° C.; and for a period of from about 0.2 hour to about 20 hours; alternatively, from about 0.5 hour to about 20 hours; alternatively, from about 1 hour to about 10 hours.

In an embodiment, a method of preparing a hydrogenation catalyst further comprises contacting the supported palladium composition with an organophosphorus compound of the type described herein (e.g., phosphine oxide, phosphate, an organophosphorus compound precursor such as an phosphate or an phosphine). The contacting can be carried out in any suitable manner that will yield a hydrogenation catalyst meeting the parameters described herein such as for example by incipient wetness impregnation. Briefly, the organophosphorus compound can comprise phosphine oxide which is dissolved in a solvent, such as for example, water, acetone, isopropanol, etc., to form a phosphine oxide containing solution. The phosphine oxide containing solution can be added to the supported palladium composition to form a palladium/phosphine oxide supported composition (herein this particular embodiment of the hydrogenation catalyst is referred to as a Pd/PO composition).

In some embodiments, one or more selectivity enhancers of the type described previously herein can be added to the supported palladium composition prior to or following the contacting of same with an organophosphorus compound. In an embodiment, this addition can occur by soaking the supported palladium composition (with or without the organophosphorus compound) in a liquid comprising one or more suitable selectivity enhancers. In another embodiment, this addition can occur by incipient wetness impregnation of the supported palladium composition (with or without an organophosphorus compound) with liquid comprising one or more suitable selectivity enhancers to form an enhanced supported palladium composition.

In an embodiment, silver can be added to the supported palladium composition (without an organophosphorus compound). For example, the supported palladium composition can be placed in an aqueous silver nitrate solution of a quantity greater than that necessary to fill the pore volume of the composition. The resulting material is a palladium/silver supported composition (herein this particular embodiment of the hydrogenation catalyst is referred to as a Pd/Ag composition). In an embodiment, the Pd/Ag composition is further contacted with an organophosphorus compound. The contacting can be carried out as described above to form a palladium/silver/phosphine oxide composition. In another embodiment, the Pd/Ag composition can be further contacted with a phosphine oxide compound (herein this particular embodiment of the hydrogenation catalyst is referred to as a Pd/Ag/PO composition).

In an embodiment, one or more alkali metals can be added to the Pd/Ag composition (prior to or following contacting with an organophosphorus compound) using any suitable technique such as those described previously herein. In an embodiment, the selectivity enhancer can comprise potassium fluoride, and the resulting material is a palladium/silver/alkali metal fluoride supported composition (herein this particular embodiment of the hydrogenation catalyst is referred to as a Pd/Ag/KF composition).

In an embodiment, the supported palladium composition can be contacted with both an alkali metal halide and a silver compound (prior to or following contacting with an organophosphorus compound). Contacting of the supported palladium composition with both an alkali metal halide and a silver compound can be carried out simultaneously; alternatively the contacting can be carried out sequentially in any user-desired order.

In an embodiment, one or more selectivity enhancers can be contacted with the supported palladium composition prior to contacting the composition with an organophosphorus compound. In such embodiments, the resulting composition comprising Pd/Ag, Pd/KF, or Pd/Ag/KF can be calcined under the conditions described previously herein, and subsequently contacted with an organophosphorus compound. For example, phosphine oxide (PO) can be added to the Pd/Ag, Pd/KF, and/or Pd/Ag/KF compositions to provide Pd/Ag/PO, Pd/KF/PO, and/or Pd/Ag/KF/PO compositions. In an alternative embodiment, one or more selectivity enhancers can be contacted with the supported palladium composition following contacting of the composition with an organophosphorus compound. For example, Ag and/or KF can be added to the Pd/PO composition to provide Pd/Ag/PO, Pd/KF/PO, and/or Pd/Ag/KF/PO compositions. In yet another alternative embodiment, one or more selectivity enhancers can be contacted with the palladium supported composition and an organophosphorus compound simultaneously.

In an embodiment, a hydrogenation catalyst formed in accordance with the methods disclosed herein can comprise an α-alumina support, palladium, and an organophosphorus compound. In an alternative embodiment, a hydrogenation catalyst formed in accordance with the methods disclosed herein can comprise an α-alumina support, palladium, an organophosphorus compound (e.g., phosphine oxide) and one or more selectivity enhancers, (e.g., silver and/or potassium fluoride). The hydrogenation catalyst (Pd/PO, Pd/Ag/PO, Pd/KF/PO, and/or the Pd/Ag/KF/PO compositions) can be dried to form a dried hydrogenation catalyst. In some embodiments, this drying step can be carried out at a temperature in the range of from about 0° C. to about 150° C.; alternatively, from about 30° C. to about 100° C.; alternatively, from about 50° C. to about 80° C.; and for a period of from about 0.1 hour to about 100 hours; alternatively, from about 0.5 hour to about 20 hours; alternatively, from about 1 hour to about 10 hours. In an embodiment, the organophosphorus compound can comprise an organophosphorus compound precursor which upon exposure to air and/or the temperature ranges used during drying of the aforementioned composition can be converted to an organophosphorus compound of the type described herein.

The dried hydrogenation catalyst can be reduced using hydrogen gas or a hydrogen gas containing feed, e.g., the feed stream of the process, thereby providing for optimum operation of the process. Such a gaseous hydrogen reduction can be carried out at a temperature in the range of from, for example, about 0° C. to about 150° C.; alternatively, 10° C. to about 100° C.; alternatively, about 20° C. to about 80° C. Additionally or alternatively, the dried hydrogenation catalyst can be reduced in a pressurized atmosphere and at a disclosed temperature, such as ambient temperature for a period of about 8 to about 24 hours.

In an embodiment, a method of preparing a hydrogenation catalyst can comprise contacting an inorganic support with a palladium-containing compound (e.g., palladium chloride, palladium nitrate) to form a palladium supported composition; drying and calcining the palladium supported composition to form a dried and calcined palladium supported composition. The dried and calcined palladium supported composition can then be contacted with a silver-containing compound (e.g., silver nitrite, silver fluoride) to form a Pd/Ag composition which can then be dried and/or calcined to form a dried and/or calcined Pd/Ag composition. The dried and/or calcined Pd/Ag composition can be contacted with an alkali metal fluoride (e.g., potassium fluoride) to form a Pd/Ag/KF composition which can then be dried and calcined. The dried and calcined Pd/Ag/KF composition can then be contacted with an organophosphorus compound (e.g., phosphine oxide or precursor) to form a hydrogenation catalyst. In an alternative embodiment, the Pd/Ag/KF composition can be added to an unsaturated hydrocarbon and the organophosphorus compound can be separately added to the unsaturated hydrocarbon so that the Pd/Ag/KF composition contacts the organophosphorus compound to form the hydrogenation catalyst while in contact with the unsaturated hydrocarbon. The hydrogenation catalyst can be further processed by drying the hydrogenation catalyst to form a dried hydrogenation catalyst. The contacting, drying, and calcining can be carried out using any suitable technique and conditions such as those described previously herein.

Examples of suitable hydrogenation catalysts and methods for preparation thereof are disclosed U.S. Pat. No. 5,489,565, U.S. Pat. No. 5,585,318, U.S. Pat. No. 5,510,550, and U.S. Patent Application Publication No. 2010/0228065 A1, each of which is incorporated herein by reference in its entirety for all purposes.

Embodiments of the disclosed process can be described using FIGS. 2 and 3. The process can include feeding or flowing a hydrocarbon via a feed stream 12 to a furnace 10; thermally cracking the hydrocarbon in the furnace 10; flowing a cracked gas stream 14 from the furnace 10 to a fractionation zone 20 or directly to the first reaction zone 30, second reaction zone 35, or both; when flowing cracked gas stream 14 to the fractionation zone 20, separating the cracked gas stream 14 into: i) a C₂⁻ stream 24 and a C₃⁺ stream 22 for frontend deethanizer configurations, ii) a C₃⁻ stream 24 and a C₄⁺ stream 22 for frontend depropanizer configurations, or iii) a C₂ stream 24 (without methane, hydrogen, and carbon monoxide) and a C₃⁺ stream 22 for backend configurations; providing (e.g., using for the purpose of hydrogenation) a first reaction zone 30 comprising a first hydrogenation catalyst and a second reaction zone 35 comprising a second hydrogenation catalyst; feeding a highly unsaturated hydrocarbon contained in hydrocarbon stream 24 (e.g., a C₂⁻ stream 24, C₃⁻ stream 24, or a C₂⁻ stream 24 (without hydrogen, carbon monoxide, and methane)) to the first reaction zone 30; hydrogenating, in the first reaction zone 30, the highly unsaturated hydrocarbon to yield one or more compounds such as an unsaturated hydrocarbon, a saturated hydrocarbon, and an unconverted highly saturated hydrocarbon, wherein conversion of the highly unsaturated hydrocarbon to the unsaturated hydrocarbon and the saturated hydrocarbon after hydrogenation in the first reaction zone 30 is about 90 mol % or greater based on moles of the highly unsaturated hydrocarbon provided to the first reaction zone 30; hydrogenating, in the second reaction zone 35, unconverted or unreacted highly unsaturated hydrocarbon to yield one or more compounds such as the unsaturated hydrocarbon, the saturated hydrocarbon, and optionally the unconverted highly unsaturated hydrocarbon, wherein a total conversion of the highly unsaturated hydrocarbon to the one or more compounds such as the unsaturated hydrocarbon and the saturated hydrocarbon is about 99 mol % or greater based on moles of the highly unsaturated hydrocarbon provided to the first reaction zone 30; or combinations thereof. At least one of the first hydrogenation catalyst and the second hydrogenation catalyst can comprise a hydrogenation selectivity to the unsaturated hydrocarbon of about 90 mol % or greater based on moles of the highly unsaturated hydrocarbon which are converted. Hydrogenation in the first reaction zone 30, the second reaction zone 35, or both can comprise contacting the first hydrogenation catalyst, the second hydrogenation catalyst, or both with the highly unsaturated hydrocarbon in the presence of hydrogen (e.g., hydrogen can be included in the hydrocarbon stream 24 and/or a separate supply of hydrogen can be fed to the first reaction zone 30, the second reaction zone 35, or both, by techniques disclosed herein). Contacting the first hydrogenation catalyst, the second hydrogenation catalyst, or both with the highly unsaturated hydrocarbon in the presence of hydrogen can be conducted at a temperature less than about the boiling point of an organophosphorus compound of the first hydrogenation catalyst, the second hydrogenation catalyst, or both.

### EXAMPLES

The disclosure having been generally described, the following examples are given to further explain the embodiments of the disclosure and to demonstrate the practice and advantages thereof. It is understood that these examples are given by way of illustration and is not intended to limit the specification or the claims in any manner.

### Example 1

A commercial process simulator was employed to generate kinetic model data in accordance with the systems and/or methods disclosed herein. The models employed an adiabatic multi-reactor system with which hydrogenates acetylene to ethylene with inter-bed coolers. The tuning factor in the kinetic model scales the rate constant of each reaction to model its declining rate with catalyst deactivation at a given temperature. The tuning factor magnitude is indicative of the activity of the hydrogenation catalyst at any given time from the startup of the catalyst; higher values indicate a more active catalyst. Table 1 summarizes the kinetic model data for the first reactor:

TABLE 1

| | Kinetic Mode No. | | | | | | | | | |
|---|---|---|---|---|---|---|---|---|---|---|
| | 1 | 2 | 3 | 4 | 5 | 6 | 7 | 8 | 9 | 10 |
| Tuning | 1.050 | 0.980 | 0.900 | 1.050 | 0.980 | 0.900 | 1.050 | 0.900 | 1.050 | 0.900 |
| Factor | | | | | | | | | | |
| (C₂H₂ + H₂ | | | | | | | | | | |
| → C₂H₄) | | | | | | | | | | |
| Tuning | 0.827 | 0.735 | 0.635 | 0.827 | 0.735 | 0.635 | 0.855 | 0.670 | 0.795 | 0.605 |
| Factor | | | | | | | | | | |
| (C₂H₄ + H₂ | | | | | | | | | | |
| → C₂H₆) | | | | | | | | | | |
| Overall | 90.09 | 90.55 | 90.61 | 90.38 | 89.89 | 90.44 | 90.62 | 90.03 | 90.17 | 90.17 |
| Selectivity | | | | | | | | | | |
| (mol %) | | | | | | | | | | |
| First | 97.0 | 97.5 | 97.5 | 97.0 | 97.5 | 97.5 | 91.0 | 94.5 | 97.5 | 97.8 |
| Reactor | | | | | | | | | | |
| Conversion | | | | | | | | | | |
| (mol %) | | | | | | | | | | |
| Effluent | 0.40 | 0.32 | 0.39 | 0.37 | 0.25 | 0.41 | 0.36 | 0.40 | 0.39 | 0.35 |
| Acetylene | | | | | | | | | | |
| (ppm) | | | | | | | | | | |
| Pressure | 551 | 551 | 551 | 300 | 300 | 551 | 551 | 551 | 551 | 551 |
| (psig) | | | | | | | | | | |
| Gas | 5519 | 5519 | 5519 | 2854 | 7774 | 7774 | 5519 | 5519 | 5519 | 5519 |
| Hourly | | | | | | | | | | |
| Space | | | | | | | | | | |
| Velocity | | | | | | | | | | |
| (GHSV) | | | | | | | | | | |
| Inlet | 0.2783 | 0.2783 | 0.2783 | 0.2783 | 0.2783 | 0.2783 | 0.5567 | 0.5567 | 0.1392 | 0.1392 |
| Acetylene | | | | | | | | | | |
| (mol %) | | | | | | | | | | |
| Inlet | 38.35 | 38.35 | 38.35 | 38.35 | 38.35 | 38.35 | 38.35 | 38.35 | 38.35 | 38.35 |
| Ethylene | | | | | | | | | | |
| (mol %) | | | | | | | | | | |
| Inlet | 0.0073 | 0.0073 | 0.0073 | 0.0073 | 0.0073 | 0.0073 | 0.0145 | 0.0145 | 0.0036 | 0.0036 |
| Acetylene: | | | | | | | | | | |
| Ethylene | | | | | | | | | | |
| Mol Ratio | | | | | | | | | | |

According to the above data in Table 1, conversion of acetylene in the first reactor ranges from 91 mol % to 97.8 mol % while hydrogenation selectivity of acetylene to ethylene ranges from 89.89 mol % to 90.61 mol %. The effluent concentration of acetylene ranges from 0.25 to 0.41 ppm by weight of the effluent. A lower acetylene concentration in the effluent does not significantly affect the ability to have conversion at about 90 mol % or greater and selectivity at about 90 mol % or greater. Moreover, the data in Table 1 demonstrates the conversion of about 90 mol % or greater and selectivity at about 90 mol % or greater is achievable for various GHSVs and pressures. In scenarios where the required conversion is not 100 mol %, the data of Table 1 demonstrates one reactor can achieve high conversion and selectivity.

### Example 2

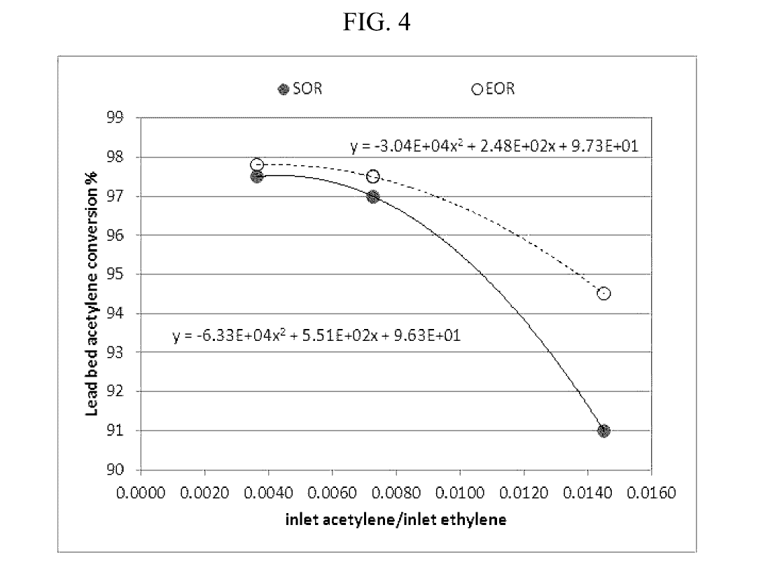

Using the commercial process simulator and kinetic model of Example 1, it was found the inlet acetylene to ethylene mole ratio of a hydrogenation reactor has some effect on conversion of acetylene. FIG. 4 shows the inlet acetylene to ethylene mole ratio in the first reactor of the kinetic model and its effect on hydrogenation conversion of acetylene in the first reactor of the kinetic model. A selectivity of 90 mol %, a tuning factor of 1.05 for the start of run (SOR) conditions, and a tuning factor of 0.90 for the end of run (EOR) conditions were used in the simulation. As can be seen in FIG. 4, conversion decreases for SOR conditions from above 97 mol % to about 91 mol % as the inlet acetylene to ethylene mole ratio increases from less than 0.0040 to above 0.0140. The conversion decreases for EOR conditions from about 98 mol % to above 94 mol % as the inlet acetylene to ethylene mole ratio increases from less than 0.0040 to above 0.0140.

Various benefits and advantages can be achieved with the disclosed embodiments.

For example, the hydrogenation catalysts disclosed herein can be used for hydrogenation of a highly unsaturated hydrocarbon at high conversions in the first reaction zone 30 and second reaction zone 35 without sacrificing catalyst selectivity (e.g., selectivity can be greater than about 90 mol % for all conversion embodiments). As such, the disclosed embodiments allow near 100 mol % conversion of the highly unsaturated hydrocarbon from the hydrocarbon stream 24 in disclosed embodiments.

Moreover, embodiments having and/or using a front-end deethanizer (e.g., fractionation zone 20) can be used in processes and systems having a process stream comprising large amounts of the saturated hydrocarbon (e.g., cracked gas stream 14). In such processes and systems, alkynes heavier than acetylene cannot be fed to the first reaction zone 30, and as such, first reaction zone 30 operate at high conversions without the risk of runaway reactions associated with streams of other compositions.

Additionally, the hydrogenation catalyst comprising an embodiment of the organophosphorus compound can display an increased activity over some time period and enhanced initial selectivity wherein the organophosphorus compound is associated with the hydrogenation catalyst. This can be advantageous for reactions employing a fresh catalyst as the organophosphorus compound can allow for a more stable operation and a reduction in the potential for a runaway reaction due to the increase in catalyst selectivity and predictable catalytic activity as the composition stabilizes.

Further, the disclosed embodiments can provide for an enhanced operating window. An operating window (ΔT) is defined as the difference between a runaway temperature (T2) at which 3 wt. % of unsaturated hydrocarbon is hydrogenated to saturated hydrocarbon from a feed stream comprising the highly unsaturated hydrocarbon and the unsaturated hydrocarbon, and the cleanup temperature (T1). Herein, the cleanup temperature is referred to as T1 and refers to the temperature at which the acetylene concentration drops below a value of about 0.3 ppmw to about 20 ppmw in the effluent when processing a representative frontend deethanizer, frontend depropanizer, or raw gas acetylene removal unit feed stream comprising the unsaturated hydrocarbon and the highly unsaturated hydrocarbon such as acetylenes and diolefins. Determinations of T1 are described in more detail for example in U.S. Pat. Nos. 7,417,007 and 6,417,136, each of which are incorporated herein in their entirety. ΔT is a convenient measure of the catalyst selectivity and operation stability in the hydrogenation of the highly unsaturated hydrocarbon (e.g., acetylene) to the unsaturated hydrocarbon (e.g., ethylene). The more selective a catalyst, the higher the temperature beyond T1 required to hydrogenate a given unsaturated hydrocarbon (e.g., ethylene). The T2 is coincident with the temperature at which a high probability of runway ethylene hydrogenation reaction could exist in an adiabatic reactor. Therefore, a larger ΔT translates to a more selective catalyst and a wider operating window for the hydrogenation of the highly unsaturated hydrocarbon to the unsaturated hydrocarbon. In an embodiment, embodiments of the hydrogenation catalyst of disclosed herein can have an operating window of from about 35° F. to about 120° F.; alternatively, from about 40° F. to about 80° F.; alternatively, from about 45° F. to about 60° F. The operating window of embodiments of the hydrogenation catalyst can be increased by greater than about 10%; alternatively, greater than about 15%; alternatively, greater than about 20% when compared to an otherwise similar catalyst prepared in the absence of an organophosphorus compound.

Additionally, the disclosed embodiments can reduce the amount of heavy side-products produced in the hydrogenation reaction. Heavy side-products can comprise molecules having four or more carbon atoms per molecule. Hydrogenation catalysts can produce heavy side-products by oligomerizing the highly unsaturated hydrocarbon (e.g., acetylene) that are present in the feed stream (e.g., hydrocarbon stream 24). The presence of heavy side-products is one of a number of contributors to the fouling of the hydrogenation catalyst that can result in catalyst deactivation. The deactivation of the hydrogenation catalyst results in the catalyst having a lower activity and selectivity to the unsaturated hydrocarbon. Embodiments of the hydrogenation catalyst described herein can exhibit a reduction in the weight percent of C₄⁺ produced at T1 of from about 1 wt. % to about 25 wt. %; alternatively, from about 1.5 wt. % to about 20 wt. %; alternatively, from about 2 wt. % to about 15 wt. %.

Additionally still, in embodiments where the hydrocarbon stream 32 can comprise a highly unsaturated hydrocarbon and an unsaturated hydrocarbon, the disclosed systems and processes provide for the selectivity and conversion levels disclosed herein when the mole ratio of highly unsaturated hydrocarbon to unsaturated hydrocarbon in the hydrocarbon stream 24 is less than about 0.0160 at start of run (e.g., based on mole percent of each component); when the ratio of highly unsaturated hydrocarbon to unsaturated hydrocarbon in the hydrocarbon stream 24 is less than about 0.021 at end of run (e.g., based on mole percent of each component); when the highly unsaturated hydrocarbon can comprise less than about 20, 19, 18, 17, 16, 15, 14, 13, 12, 11, 10, 9, 8, 7, 6, 5, 4, 3, 2, 1 or less wt. % of the hydrocarbon stream 24; or combinations thereof.

### ADDITIONAL DESCRIPTION

Embodiments of a system and process have been described. The following are a first set of nonlimiting, specific embodiments in accordance with the present disclosure:

A first embodiment, which is a process comprising:

hydrogenating a highly unsaturated hydrocarbon in the presence of a first hydrogenation catalyst to yield an unsaturated hydrocarbon, a saturated hydrocarbon, and an unconverted highly unsaturated hydrocarbon, wherein a conversion of the highly unsaturated hydrocarbon to the unsaturated hydrocarbon and the saturated hydrocarbon in the presence of the first hydrogenation catalyst is about 90 mol % or greater; and

hydrogenating the unconverted highly unsaturated hydrocarbon in the presence of a second hydrogenation catalyst to yield the unsaturated hydrocarbon and the saturated hydrocarbon, and the unconverted highly unsaturated hydrocarbon, wherein a total conversion of the highly unsaturated hydrocarbon to the unsaturated hydrocarbon and the saturated hydrocarbon after hydrogenation in the presence of the first hydrogenation catalyst and after hydrogenation in the presence of the second hydrogenation catalyst is about 99 mol % or greater;

wherein the first hydrogenation catalyst, the second hydrogenation catalyst, or both, have a hydrogenation selectivity to the unsaturated hydrocarbon of about 90% or greater based on the moles of the highly unsaturated hydrocarbon which are converted.

A second embodiment, which is the process of the first embodiment, further comprising:

flowing an effluent stream comprising the unsaturated hydrocarbon, the saturated hydrocarbon, and the unconverted highly unsaturated hydrocarbon from the first hydrogenation catalyst to the second hydrogenation catalyst, wherein no heat is added to the effluent stream.

A third embodiment, which is the process of any of the first through the second embodiments, further comprising:

flowing an effluent stream comprising the unsaturated hydrocarbon, the saturated hydrocarbon, and the unconverted highly unsaturated hydrocarbon from the first hydrogenation catalyst to the second hydrogenation catalyst,

wherein a first temperature of the effluent stream as the effluent stream flows into the second hydrogenation catalyst is the same as or lower than a second temperature of the effluent stream as the effluent stream flows from the first hydrogenation catalyst.

A fourth embodiment, which is the process of any of the first through the third embodiments, wherein the highly unsaturated hydrocarbon comprises acetylene, wherein the unsaturated hydrocarbon comprise ethylene, and wherein the saturated hydrocarbon comprises ethane.

A fifth embodiment, which is the process of any of the first through the fourth embodiments, wherein the highly unsaturated hydrocarbon comprises methylacetylene, propadiene, or both; wherein the unsaturated hydrocarbon comprises propylene; and wherein the saturated hydrocarbon comprises propane.

A sixth embodiment, which is the process of any of the first through the fifth embodiments, further comprising:

cracking a feed stream to produce a cracked gas stream comprising the highly unsaturated hydrocarbon, the unsaturated hydrocarbon, and the saturated hydrocarbon.

A seventh embodiment, which is the process of the sixth embodiment, wherein the cracked gas stream comprises from about 10 ppm to about 20,000 ppm of the highly unsaturated hydrocarbon based on the total weight of all hydrocarbons in the cracked gas stream.

An eighth embodiment, which is the process of any of the sixth through the seventh embodiments, further comprising:

fractionating the cracked gas stream to yield a C₂⁻ stream comprising the highly unsaturated hydrocarbon, the unsaturated hydrocarbon, and the saturated hydrocarbon, wherein at least a portion of the highly unsaturated hydrocarbon in the C₂⁻ stream is hydrogenated in the presence of the first and the second hydrogenation catalysts.

A ninth embodiment, which is the process of any of the first through the eighth embodiments, further comprising:

separating the unsaturated hydrocarbon from the saturated hydrocarbon after hydrogenation of the highly unsaturated hydrocarbon.

A tenth embodiments, which is the process of any of the sixth through the seventh embodiments, further comprising:

fractionating the cracked gas stream to yield a C₃⁻ stream comprising the highly unsaturated hydrocarbon, the unsaturated hydrocarbon, and the saturated hydrocarbon, wherein at least a portion of the highly unsaturated hydrocarbon in the C₃⁻ stream is hydrogenated in the presence of the first and the second hydrogenation catalysts.

An eleventh embodiment, which is the process of any of the sixth through the seventh embodiments, further comprising:

fractionating the cracked gas stream to yield a C₂⁺ stream comprising the highly unsaturated hydrocarbon, the unsaturated hydrocarbon, and the saturated hydrocarbon; and

fractionating the C₂⁺ stream to yield a C₂⁻ stream comprising the highly unsaturated hydrocarbon, the unsaturated hydrocarbon, and the saturated hydrocarbon, wherein at least a portion of the highly unsaturated hydrocarbon in the C₂⁻ stream is hydrogenated in the presence of the first and the second hydrogenation catalysts.

A twelfth embodiment, which is the process of any of the sixth through the eleventh embodiments, wherein at least a portion of the highly unsaturated hydrocarbon in the cracked gas stream is hydrogenated in the presence of the first and the second hydrogenation catalysts.

A thirteenth embodiment, which is the process of any of the first through the twelfth embodiments, wherein the step of hydrogenating the highly unsaturated hydrocarbon comprises:

contacting the first hydrogenation catalyst with at least a portion of the highly unsaturated hydrocarbon in the presence of hydrogen;

wherein the step of hydrogenating the unconverted highly saturated hydrocarbon comprises:

contacting the second hydrogenation catalyst with at least a portion of the unconverted highly unsaturated hydrocarbon in the presence of hydrogen.

A fourteenth embodiment, which is the process of any of the first through the thirteenth embodiments, wherein the at least one of the first hydrogenation catalyst and the second hydrogenation catalyst comprises palladium and an inorganic support.

A fifteenth embodiment, which is the process of the fourteenth embodiment, wherein the inorganic support has a surface area of from about 2 m²/g to about 100 m²/g, and greater than about 90 wt. % of the palladium is concentrated near a periphery of the inorganic support.

A sixteenth embodiment, which is the process of any of the fourteenth through the fifteenth embodiments, wherein the at least one of the first hydrogenation catalyst and the second hydrogenation catalyst further comprises Group 1B metals, Group 1B metal compounds, silver compounds, fluorine, fluoride compounds, sulfur, sulfur compounds, alkali metal, alkali metal compounds, alkaline earth metals, alkaline earth metal compounds, iodine, iodide compounds, or combinations thereof.

A seventeenth embodiment, which is the process of any of the fourteenth through the sixteenth embodiments, wherein the palladium is present in the first hydrogenation catalyst or second hydrogenation catalyst in an amount of from about 0.005 wt. % to about 5 wt. % based on the total weight of the first hydrogenation catalyst or second hydrogenation catalyst.

An eighteenth embodiment, which is the process of any of the fourteenth through the seventeenth embodiments, wherein the inorganic support comprises an alpha alumina support.

A nineteenth embodiment, which is the process of any of the fourteenth through the eighteenth embodiments, wherein the inorganic support comprises a chloride-treated alpha alumina support.

A twentieth embodiment, which is the process of any of the fourteenth through the nineteenth embodiments, wherein at least one of the first hydrogenation catalyst and the second hydrogenation catalyst further comprises an organophosphorus compound.

An twenty-first embodiment, which is the process of the twentieth embodiment, wherein the organophosphorus compound of the hydrogenation catalyst is:

i) present in an amount of from about 0.005 wt. % to about 5 wt. % based on the total weight of the hydrogenation catalyst;

ii) represented by a general formula (R)ₓ(OR′)_{y}P═O, wherein x and y are integers ranging from 0 to 3 and x plus y equals 3, wherein each R is hydrogen, a hydrocarbyl group, or combinations thereof; and wherein each R′ is a hydrocarbyl group;

iii) a product of an organophosphorus compound precursor represented by the general formula of (R)ₓ(OR′)_{y}P, wherein x and y are integers ranging from 0 to 3 and x plus y equals 3, wherein each R is hydrogen, a hydrocarbyl group, or combinations thereof; and wherein each R′ is a hydrocarbyl group;

iv) a phosphine oxide, a phosphate, a phosphinate, a phosphonate, a phosphine, a phosphite, a phosphinite, a phosphonite, or combinations thereof; or

v) combinations thereof.

A twenty-second embodiment, which is a system comprising:

a hydrocarbon stream comprising a highly unsaturated hydrocarbon, an unsaturated hydrocarbon, and optionally, a saturated hydrocarbon;

a first reaction zone comprising a first hydrogenation catalyst, wherein the hydrocarbon stream contacts the first hydrogenation catalyst in the first reaction zone, and wherein at least a portion of the highly unsaturated hydrocarbon from the hydrocarbon stream is hydrogenated in the first reaction zone; and

a second reaction zone comprising a second hydrogenation catalyst, wherein the second reaction zone receives a first effluent stream comprising the unsaturated hydrocarbon, an unconverted highly unsaturated hydrocarbon, and optionally, the saturated hydrocarbon from the first reaction zone, wherein at least a portion of the unconverted highly unsaturated hydrocarbon is hydrogenated in the second reaction zone;

wherein conversion of the highly unsaturated hydrocarbon to the unsaturated hydrocarbon and the saturated hydrocarbon after hydrogenation in the first reaction zone is about 90 mol % or greater based on moles of the highly unsaturated hydrocarbon in the hydrocarbon stream,

wherein a total conversion of the highly unsaturated hydrocarbon to the unsaturated hydrocarbon and the saturated hydrocarbon after hydrogenation in the first and the second reaction zones is about 99 mol % or greater based on moles of the highly unsaturated hydrocarbon in the hydrocarbon stream, and

wherein the first hydrogenation catalyst, the second hydrogenation catalyst, or both have a hydrogenation selectivity to the unsaturated hydrocarbon of about 90 mol % or greater based on the moles of highly unsaturated hydrocarbon which are converted.

A twenty-third embodiment, which is the system of the twenty-second embodiment, further comprising:

a first effluent stream comprising the unsaturated hydrocarbon, the saturated hydrocarbon, and the unconverted highly unsaturated hydrocarbon, wherein the first effluent stream flows from the first reaction zone to the second reaction zone, wherein no heat is added to the first effluent stream between the first reaction zone and the second reaction zone.

A twenty-fourth embodiment, which is the system of any of the twenty-second through the twenty-third embodiments, further comprising:

a first effluent stream comprising the unsaturated hydrocarbon, the saturated hydrocarbon, and the unconverted highly unsaturated hydrocarbon, wherein the first effluent stream flows from the first reaction zone to the second reaction zone, wherein a first temperature of the first effluent stream as the first effluent stream flows into the second reaction zone is the same as or lower than a second temperature of the first effluent stream as the first effluent stream flows from the first reaction zone.

A twenty-fifth embodiment, which is the system of any of the twenty-second through the twenty-fourth embodiments, further comprising:

a cracked gas stream comprising ethane; and

a fractionation zone upstream of the first reaction zone to fractionate the cracked gas stream into an overhead product and a bottoms product, wherein the overhead product comprises about 90 mol % or greater of the ethane contained in the cracked gas stream, wherein the overhead product is fed to the first reaction zone via the hydrocarbon stream.

A twenty-sixth embodiment, which is the system of any of the twenty-second through the twenty-fourth embodiments, further comprising:

a cracked gas stream comprising ethane and methane; and

a fractionation zone upstream of the first reaction zone to fractionate the cracked gas stream into an overhead product and a bottoms product, wherein the overhead product is a methane-rich stream, wherein the bottoms product comprises about 90 mol % or greater of the ethane contained in the cracked gas stream, wherein the bottoms product is fed to the first reaction zone via the hydrocarbon stream.

A twenty-seventh embodiment, which is the system of any of the twenty-second through the twenty-sixth embodiments, further comprising:

a feed stream; and

a furnace upstream of the first reaction zone to crack the feed stream so as to yield a cracked gas stream comprising hydrogen, carbon monoxide, propane, ethane, methane, methylacetylene, propadiene, acetylene, ethylene, propylene, C₄⁺ components, or combinations thereof, wherein the cracked gas stream is fed to the first reaction zone via the hydrocarbon stream.

A twenty-eighth embodiment, which is the system of any of the twenty-second through the twenty-seventh embodiments, further comprising:

a fractionation zone downstream of the second reaction zone, wherein the fractionation zone separates the unsaturated hydrocarbon from the saturated hydrocarbon.

A twenty-ninth embodiment, which is the system of any of the twenty-second through the twenty-eighth embodiments, wherein the highly unsaturated hydrocarbon comprises acetylene, wherein the unsaturated hydrocarbon comprises ethylene, wherein the saturated hydrocarbon comprises ethane.

A thirtieth embodiment, which is the system of any of the twenty-second through the twenty-ninth embodiments, wherein at least one of the first hydrogenation catalyst and the second hydrogenation catalyst comprises palladium and an inorganic support.

A thirty-first embodiment, which is the system of the thirtieth embodiment, wherein the inorganic support has a surface area of from about 2 m²/g to about 100 m²/g, and greater than about 90 wt. % of the palladium is concentrated near a periphery of the inorganic support.

A thirty-second embodiment, which is the system of any of the thirtieth through the thirty-first embodiments, wherein at least one of the first hydrogenation catalyst and the second hydrogenation catalyst further comprises Group 1B metals, Group 1B metal compounds, silver compounds, fluorine, fluoride compounds, sulfur, sulfur compounds, alkali metal, alkali metal compounds, alkaline earth metals, alkaline earth metal compounds, iodine, iodide compounds, or combinations thereof.

A thirty-third embodiment, which is the system of any of the thirtieth through the thirty-second embodiments, wherein the palladium is present in an amount of from about 0.005 wt. % to about 5 wt. % based on the total weight of the catalyst.

A thirty-fourth embodiment, which is the system of any of the thirtieth through the thirty-third embodiments, wherein the inorganic support comprises a chloride-treated alpha alumina support.

A thirty-fifth embodiment, which is the system of any of the thirtieth through the thirty-fourth embodiments, wherein at least one of the first hydrogenation catalyst and the second hydrogenation catalyst further comprises an organophosphorus compound.

A thirty-sixth embodiment, which is the system of the thirty-fifth embodiment, wherein the first reaction zone, the second reaction zone, or both, operate at a temperature less than about the boiling point of the organophosphorus compound.

A thirty-seventh embodiment, which is the system of any of the thirty-fifth through the thirty-sixth embodiments, wherein the organophosphorus compound of the hydrogenation catalyst is:

i) present in an amount of from about 0.005 wt. % to about 5 wt. % based on the total weight of the hydrogenation catalyst;

ii) represented by a general formula (R)ₓ(OR′)_{y}P═O, wherein x and y are integers ranging from 0 to 3 and x plus y equals 3, wherein each R is hydrogen, a hydrocarbyl group, or combinations thereof; and wherein each R′ is a hydrocarbyl group;

iii) a product of an organophosphorus compound precursor represented by the general formula of (R)ₓ(OR′)_{y}P, wherein x and y are integers ranging from 0 to 3 and x plus y equals 3, wherein each R is hydrogen, a hydrocarbyl group, or combinations thereof; and wherein each R′ is a hydrocarbyl group;

iv) a phosphine oxide, a phosphate, a phosphinate, a phosphonate, a phosphine, a phosphite, a phosphinite, a phosphonite, or combinations thereof; or

v) combinations thereof.

A thirty-eighth embodiment, which is a process comprising:

cracking a feed stream to produce a cracked gas stream comprising acetylene, ethylene, ethane, methane, hydrogen, carbon monoxide, and C₃⁺ components;

hydrogenating acetylene in the presence of a first hydrogenation catalyst in a first reaction zone, wherein conversion of acetylene to ethylene and ethane in the first reaction zone is about 90 mol % or greater of the total acetylene in the reaction zone;

receiving a first effluent stream from the first reaction zone into a second reaction zone, wherein the first effluent stream comprises unconverted acetylene;

hydrogenating the unconverted acetylene of the first effluent stream in the presence of a second hydrogenation catalyst in the second reaction zone, wherein a total conversion of acetylene to ethylene and ethane after hydrogenation in the first reaction zone and the second reaction zone is about 99 mol % or greater of the total acetylene present in the C₂⁻-stream;

recovering a second effluent stream from the second reaction zone;

removing ethylene from the second effluent stream to yield an ethylene stream; and

polymerizing ethylene from the ethylene stream into one or more polymer products;

wherein the first hydrogenation catalyst, the second hydrogenation catalyst, or both, have a hydrogenation selectivity to ethylene of about 90 mol % or greater based on moles of acetylene which are converted.

A thirty-ninth embodiment, which is the process of the thirty-eighth embodiment, further comprising:

fractionating the cracked gas stream into a C₂⁻ stream and a C₃⁺ stream, wherein the C₂⁻ stream comprises acetylene, ethylene, ethane, and methane, wherein the C₃⁺ stream comprises the C₃⁺ components; and

feeding the C₂⁻ stream to the first reaction zone.

A fortieth embodiment, which is the process of the thirty-eighth embodiment, further comprising:

fractionating the cracked gas stream into a C₃⁻ stream and a C₄⁺ stream, wherein the C₃⁻ stream comprises methylacetylene, propadiene, propylene, acetylene, ethylene, ethane, and methane, wherein the C₃⁺ stream comprises the C₃⁺ components; and

feeding the C₃⁻ stream to the first reaction zone.

A forty-first embodiment, which is the process of the thirty-eighth embodiment, further comprising:

fractionating the cracked gas stream into a C₂⁺ stream and a methane-rich stream, wherein the methane-rich stream comprises methane, hydrogen, and carbon monoxide, wherein the C₂⁺ stream comprises methylacetylene, propadiene, propylene, acetylene, ethylene, and ethane; and fractionating the C₂⁺ stream into a C₂⁻ stream and a C₃⁺ stream, wherein the C₂⁻ stream comprises acetylene, ethylene, and ethane; and

feeding the C₂⁻ stream to the first reaction zone.

A forty-second embodiment, which is the process of the thirty-eighth embodiment, further comprising:

feeding the cracked gas stream directly to the first reaction zone.

A forty-third embodiment, which is the process of any of the thirty-eighth to the forty-second embodiments, wherein at least one of the first hydrogenation catalyst and the second hydrogenation catalyst comprises palladium and an inorganic support.

A forty-fourth embodiment, which is the process of any of the thirty-eighth to the forty-second embodiments, wherein at least one of the first hydrogenation catalyst and the second hydrogenation catalyst further comprises an organophosphorus compound.

A forty-fifth embodiment, which is a process comprising:

providing a first reaction zone comprising a first hydrogenation catalyst and a second reaction zone comprising a second hydrogenation catalyst, wherein the second reaction zone is fluidly connected to and downstream of the first reaction zone, wherein at least one of the first hydrogenation catalyst and the second hydrogenation catalyst comprises a hydrogenation catalyst, and optionally, an organophosphorus compound;

providing a highly unsaturated hydrocarbon to the first reaction zone;

hydrogenating, in the first reaction zone, the highly unsaturated hydrocarbon to yield an unsaturated hydrocarbon, a saturated hydrocarbon, and an unconverted highly unsaturated hydrocarbon, wherein conversion of the highly unsaturated hydrocarbon to the unsaturated hydrocarbon and the saturated hydrocarbon after hydrogenation in the first reaction zone is about 90 mol % or greater based on moles of the highly unsaturated hydrocarbon provided to the first reaction zone; and

hydrogenating, in the second reaction zone, the unconverted highly unsaturated hydrocarbon to yield the unsaturated hydrocarbon and the saturated hydrocarbon, wherein a total conversion of the highly unsaturated hydrocarbon to the unsaturated hydrocarbon and the saturated hydrocarbon after hydrogenation in the first reaction zone and the second reaction zone is about 99 mol % or greater based on moles of the highly unsaturated hydrocarbon provided to the first reaction zone;

wherein at least one of the first hydrogenation catalyst and the second hydrogenation catalyst comprises a hydrogenation selectivity to the unsaturated hydrocarbon of about 90 mol % or greater based on moles of the highly unsaturated hydrocarbon which are converted.

A forty-sixth embodiment, which is the process of the forty-fifth embodiment, wherein the first hydrogenation catalyst and the second hydrogenation catalyst are the same or different.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The disclosures of all patents, patent applications, and publications cited herein are hereby incorporated by reference, to the extent that they provide exemplary, procedural or other details supplementary to those set forth herein.

What is claimed is:

1. A process comprising:
hydrogenating a highly unsaturated hydrocarbon in the presence of a first hydrogenation catalyst to yield an unsaturated hydrocarbon, a saturated hydrocarbon, and an unconverted highly unsaturated hydrocarbon, wherein a conversion of the highly unsaturated hydrocarbon to the unsaturated hydrocarbon and the saturated hydrocarbon in the presence of the first hydrogenation catalyst is about 90 mol % or greater; and
hydrogenating the unconverted highly unsaturated hydrocarbon in the presence of a second hydrogenation catalyst to yield the unsaturated hydrocarbon and the saturated hydrocarbon, and the unconverted highly unsaturated hydrocarbon, wherein a total conversion of the highly unsaturated hydrocarbon to the unsaturated hydrocarbon and the saturated hydrocarbon after hydrogenation in the presence of the first hydrogenation catalyst and after hydrogenation in the presence of the second hydrogenation catalyst is about 99 mol % or greater;
wherein the first hydrogenation catalyst, the second hydrogenation catalyst, or both, have a hydrogenation selectivity to the unsaturated hydrocarbon of about 90% or greater based on the moles of the highly unsaturated hydrocarbon which are converted.

2. The process of claim 1, further comprising:
flowing a first effluent stream comprising the unsaturated hydrocarbon, the saturated hydrocarbon, and the unconverted highly unsaturated hydrocarbon from the first hydrogenation catalyst to the second hydrogenation catalyst, wherein no heat is added to the first effluent stream.

3. The process of claim 1, further comprising:
flowing a first effluent stream comprising the unsaturated hydrocarbon, the saturated hydrocarbon, and the unconverted highly unsaturated hydrocarbon from the first hydrogenation catalyst to the second hydrogenation catalyst,
wherein a first temperature of the first effluent stream as the first effluent stream flows into the second hydrogenation catalyst is the same as or lower than a second temperature of the first effluent stream as the first effluent stream flows from the first hydrogenation catalyst.

4. The process of claim 1, wherein the highly unsaturated hydrocarbon comprises acetylene, wherein the unsaturated hydrocarbon comprise ethylene, and wherein the saturated hydrocarbon comprises ethane.

5. The process of claim 1, wherein the highly unsaturated hydrocarbon comprises methylacetylene, propadiene, or both; wherein the unsaturated hydrocarbon comprises propylene; and wherein the saturated hydrocarbon comprises propane.

6. The process of claim 1, further comprising:
cracking a feed stream to produce a cracked gas stream comprising the highly unsaturated hydrocarbon, the unsaturated hydrocarbon, and the saturated hydrocarbon.

7. The process of claim 6, wherein the cracked gas stream comprises from about 10 ppm to about 20,000 ppm of the highly unsaturated hydrocarbon based on the total weight of all hydrocarbons in the cracked gas stream.

8. The process of claim 6, further comprising:
fractionating the cracked gas stream to yield a C₂⁻ stream comprising the highly unsaturated hydrocarbon, the unsaturated hydrocarbon, and the saturated hydrocarbon, wherein at least a portion of the highly unsaturated hydrocarbon in the C₂⁻ stream is hydrogenated in the presence of the first and the second hydrogenation catalysts.

9. The process of claim 8, further comprising:
separating the unsaturated hydrocarbon from the saturated hydrocarbon after hydrogenation of the highly unsaturated hydrocarbon.

10. The process of claim 6, further comprising:
fractionating the cracked gas stream to yield a C₃⁻ stream comprising the highly unsaturated hydrocarbon, the unsaturated hydrocarbon, and the saturated hydrocarbon, wherein at least a portion of the highly unsaturated hydrocarbon in the C₃⁻ stream is hydrogenated in the presence of the first and the second hydrogenation catalysts.

11. The process of claim 6, further comprising:
fractionating the cracked gas stream to yield a C₂⁺ stream comprising the highly unsaturated hydrocarbon, the unsaturated hydrocarbon, and the saturated hydrocarbon; and
fractionating the C₂⁺ stream to yield a C₂⁻ stream comprising the highly unsaturated hydrocarbon, the unsaturated hydrocarbon, and the saturated hydrocarbon, wherein at least a portion of the highly unsaturated hydrocarbon in the C₂⁻ stream is hydrogenated in the presence of the first and the second hydrogenation catalysts.

12. The process of claim 6, wherein at least a portion of the highly unsaturated hydrocarbon in the cracked gas stream is hydrogenated in the presence of the first and the second hydrogenation catalysts.

13. The process of claim 1, wherein the step of hydrogenating the highly unsaturated hydrocarbon comprises:
contacting the first hydrogenation catalyst with at least a portion of the highly unsaturated hydrocarbon in the presence of hydrogen;
wherein the step of hydrogenating the unconverted highly saturated hydrocarbon comprises:
contacting the second hydrogenation catalyst with at least a portion of the unconverted highly unsaturated hydrocarbon in the presence of hydrogen.

14. A system comprising:
a hydrocarbon stream comprising a highly unsaturated hydrocarbon, an unsaturated hydrocarbon, and optionally, a saturated hydrocarbon;
a first reaction zone comprising a first hydrogenation catalyst, wherein the hydrocarbon stream contacts the first hydrogenation catalyst in the first reaction zone, and wherein at least a portion of the highly unsaturated hydrocarbon from the hydrocarbon stream is hydrogenated in the first reaction zone; and
a second reaction zone comprising a second hydrogenation catalyst, wherein the second reaction zone receives a first effluent stream comprising the unsaturated hydrocarbon, an unconverted highly unsaturated hydrocarbon, and optionally, the saturated hydrocarbon from the first reaction zone, wherein at least a portion of the unconverted highly unsaturated hydrocarbon is hydrogenated in the second reaction zone;
wherein a conversion of the highly unsaturated hydrocarbon to the unsaturated hydrocarbon and the saturated hydrocarbon after hydrogenation in the first reaction zone is about 90 mol % or greater based on moles of the highly unsaturated hydrocarbon in the hydrocarbon stream,
wherein a total conversion of the highly unsaturated hydrocarbon to the unsaturated hydrocarbon and the saturated hydrocarbon after hydrogenation in the first and the second reaction zones is about 99 mol % or greater based on moles of the highly unsaturated hydrocarbon in the hydrocarbon stream, and
wherein the first hydrogenation catalyst, the second hydrogenation catalyst, or both have a hydrogenation selectivity to the unsaturated hydrocarbon of about 90 mol % or greater based on the moles of highly unsaturated hydrocarbon which are converted.

15. The system of claim 14, further comprising:
a first effluent stream comprising the unsaturated hydrocarbon, the saturated hydrocarbon, and the unconverted highly unsaturated hydrocarbon, wherein the first effluent stream flows from the first reaction zone to the second reaction zone, wherein no heat is added to the first effluent stream between the first reaction zone and the second reaction zone.

16. The system of claim 15, wherein a first temperature of the first effluent stream as the first effluent stream flows into the second reaction zone is the same as or lower than a second temperature of the first effluent stream as the first effluent stream flows from the first reaction zone.

17. The system of claim 14, further comprising:
a cracked gas stream comprising ethane; and
a fractionation zone upstream of the first reaction zone to fractionate the cracked gas stream into an overhead product and a bottoms product, wherein the overhead product comprises about 90 mol % or greater of the ethane contained in the cracked gas stream, wherein the overhead product is fed to the first reaction zone via the hydrocarbon stream.

18. The system of claim 14, further comprising:
a cracked gas stream comprising ethane and methane; and
a fractionation zone upstream of the first reaction zone to fractionate the cracked gas stream into an overhead product and a bottoms product, wherein the overhead product is a methane-rich stream, wherein the bottoms product comprises about 90 mol % or greater of the ethane contained in the cracked gas stream, wherein the bottoms product is fed to the first reaction zone via the hydrocarbon stream.

19. The system of claim 14, further comprising:
a feed stream; and
a furnace upstream of the first reaction zone to crack the feed stream so as to yield a cracked gas stream comprising hydrogen, carbon monoxide, propane, ethane, methane, methylacetylene, propadiene, acetylene, ethylene, propylene, C₄⁺ components, or combinations thereof, wherein the cracked gas stream is fed to the first reaction zone via the hydrocarbon stream.

20. The system of claim 14, further comprising:
a fractionation zone downstream of the second reaction zone, wherein the fractionation zone separates the unsaturated hydrocarbon from the saturated hydrocarbon.

21. The system of claim 14, wherein the highly unsaturated hydrocarbon comprises acetylene, wherein the unsaturated hydrocarbon comprises ethylene, wherein the saturated hydrocarbon comprises ethane.

22. A process comprising:
cracking a feed stream to produce a cracked gas stream comprising acetylene, ethylene, ethane, methane, and C₃⁺ components;
hydrogenating acetylene in the presence of a first hydrogenation catalyst in a first reaction zone, wherein conversion of acetylene to ethylene and ethane in the first reaction zone is about 90 mol % or greater of the total acetylene fed to the first reaction zone;
receiving a first effluent stream from the first reaction zone into a second reaction zone, wherein the first effluent stream comprises unconverted acetylene;
hydrogenating the unconverted acetylene of the first effluent stream in the presence of a second hydrogenation catalyst in the second reaction zone, wherein a total conversion of acetylene to ethylene and ethane after hydrogenation in the first reaction zone and the second reaction zone is about 99 mol % or greater of the total acetylene fed to the first reaction zone;
recovering a second effluent stream from the second reaction zone;
removing ethylene from the second effluent stream to yield an ethylene stream; and
polymerizing ethylene from the ethylene stream into one or more polymer products;
wherein the first hydrogenation catalyst, the second hydrogenation catalyst, or both, have a hydrogenation selectivity to ethylene of about 90 mol % or greater based on moles of acetylene which are converted.

23. The process of claim 22, further comprising:
fractionating the cracked gas stream into a C₂⁻ stream and a C₃⁺ stream, wherein the C₂⁻ stream comprises acetylene, ethylene, ethane, and methane, wherein the C₃⁺ stream comprises the C₃⁺ components; and
feeding the C₂⁻ stream to the first reaction zone.

24. The process of claim 22, further comprising:
fractionating the cracked gas stream into a C₃⁻ stream and a C₄⁺ stream, wherein the C₃⁻ stream comprises methylacetylene, propadiene, propylene, acetylene, ethylene, ethane, and methane, wherein the C₃⁺ stream comprises the C₃⁺ components; and
feeding the C₃⁻ stream to the first reaction zone.

25. The process of claim 22, further comprising:
fractionating the cracked gas stream into a C₂⁺ stream and a methane-rich stream, wherein the methane-rich stream comprises methane, hydrogen, and carbon monoxide, wherein the C₂⁺ stream comprises methylacetylene, propadiene, propylene, acetylene, ethylene, and ethane; and
fractionating the C₂⁺ stream into a C₂⁻ stream and a C₃⁺ stream, wherein the C₂⁻ stream comprises acetylene, ethylene, and ethane; and
feeding the C₂⁻ stream to the first reaction zone.

26. The process of claim 22, further comprising:
feeding the cracked gas stream directly to the first reaction zone.

27. A process comprising:
providing a first reaction zone comprising a first hydrogenation catalyst and a second reaction zone comprising a second hydrogenation catalyst, wherein the second reaction zone is fluidly connected to and downstream of the first reaction zone, wherein at least one of the first hydrogenation catalyst and the second hydrogenation catalyst comprises a hydrogenation catalyst, and optionally, an organophosphorus compound;
providing a highly unsaturated hydrocarbon to the first reaction zone;
hydrogenating, in the first reaction zone, the highly unsaturated hydrocarbon to yield an unsaturated hydrocarbon, a saturated hydrocarbon, and an unconverted highly unsaturated hydrocarbon, wherein conversion of the highly unsaturated hydrocarbon to the unsaturated hydrocarbon and the saturated hydrocarbon after hydrogenation in the first reaction zone is about 90 mol % or greater based on moles of the highly unsaturated hydrocarbon provided to the first reaction zone; and
hydrogenating, in the second reaction zone, the unconverted highly unsaturated hydrocarbon to yield the unsaturated hydrocarbon and the saturated hydrocarbon, wherein a total conversion of the highly unsaturated hydrocarbon to the unsaturated hydrocarbon and the saturated hydrocarbon after hydrogenation in the first reaction zone and the second reaction zone is about 99 mol % or greater based on moles of the highly unsaturated hydrocarbon provided to the first reaction zone;
wherein at least one of the first hydrogenation catalyst and the second hydrogenation catalyst comprises a hydrogenation selectivity to the unsaturated hydrocarbon of about 90 mol % or greater based on moles of the highly unsaturated hydrocarbon which are converted.
